(19) United States
(12) Patent Application Publication
(10) Pub. No.: US 2018/0369120 A1
(43) Pub. Date: Dec. 27, 2018

(54) FRAGRANCE DELIVERY COMPOSITION COMPRISING COPOLYMERS OF ACRYLOYL LACTAM AND ALKYLMETHACRYLATES, PROCESS FOR PREPARING THE SAME, AND METHOD OF USE THEREOF

(71) Applicant: ISP INVESTMENTS LLC, Wilmington, DE (US)

(72) Inventors: Manuel GAMEZ-GARCIA, New City, NY (US); Solomon T. WOSSENE, Riverdale, NJ (US); Michael A. TALLON, Aberdeen, NJ (US); Drupesh PATEL, Lake Hiawatha, NJ (US); Ryan Vincent BLAZEWICZ, Lake Hiawatha, NJ (US)

(73) Assignee: ISP INVESTMENTS LLC, Wilmington, DE (US)

(21) Appl. No.: 15/775,645

(22) PCT Filed: Nov. 21, 2016
(86) PCT No.: PCT/US16/63015

§ 371 Date: May 11, 2018

Related U.S. Application Data
(60) Provisional application No. 62/257,828, filed on Nov. 20, 2015.

(51) Int. Cl. A61K 8/81; C08F 220/68; A61K 8/31; A61Q 13/00; A61Q 5/00; A61Q 5/02; A61Q 5/12; C08F 220/36; C08F 220/18

(52) U.S. Cl. CPC A61K 8/8152; C08F 220/68; A61K 8/31; A61Q 13/00; A61Q 5/00; C08F 2220/1841; A61Q 5/12; C08F 220/36; C08F 220/18; A61K 2800/21; C08F 2800/20; A61Q 5/02

(57) ABSTRACT

Disclosed herein is an oil soluble composition comprising a copolymer having repeating units of: (i) about 0.1-99.9 wt. % of at least one monomer selected from at least one hydrophobically modified (alk) acrylate moiety; and (ii) about 0.1-99.9 wt. % of at least one monomer derived from at least one functionalized or unfunctionalized acryloyl moiety and at least one lactam moiety. Also, disclosed herein is a mineral oil soluble personal care and fragrance composition comprising such copolymer, a fragrance delivery system and method of delivering fragrance.

### FIELD OF THE INVENTION

The present application relates to an oil soluble compositions comprising copolymers having repeating units of a first monomer selected from at least one hydrophobically modified (alk)acrylate moiety and a second monomer having at least one functionalized or unfunctionalized acryloyl moiety and at least one lactam moiety, and further to the use of such oil soluble compositions as a delivery system for hydrophobic based functional ingredients.

### BACKGROUND OF THE INVENTION

Numerous attempts have been made for effective and controlled release of active or functional ingredients on various substrates. One such attempt relates to controlled release techniques through which the delivery of oil based functional ingredients are achieved for fragrance based compounds. Fragrance delivery in hair care products are restricted by considerations such as availability, cost, compatibility of the fragrance ingredients with other co-components in the products or composition of interest and the ability of fragrance ingredients to be deposited or adsorbed onto the hair and survive the wash and rinse process. Furthermore, a large amount of fragrance is often lost during the wash, rinse or drying process as fragrance is bound to the micelles of the surfactant, even when the hair is air-dried. The use of fragrance delivery systems comprising fragrance molecules adsorbed onto polymeric carrier materials has also been explored. However, the fragrance release rate is not consistent. Thus, an effective fragrance release system which is stable for longer is desirable.

U.S. Pat. No. 2,882,262 assigned to Eastman Kodak Company discloses N-(acryloxyalkyl)- and N-(methacryloxyalkyl)-2-pyrrolidones, polymers thereof, and a process for their preparation. The polymers are useful in the photographic art.

U.S. Pat. No. 3,406,238 assigned to GAF Corporation discloses N-Methacryloyloxyethyl pyrrolidone in toiletry and cosmetic compositions particularly for fragrance delivery for skin care.

U.S. Published Application No. 2010/0166985 assigned to BASF SE discloses aqueous dispersions of (meth)acrylic esters of polymers comprising N-hydroxyalkylated lactam units wherein the monomer copolymerized C₁-C₁₈ alkyl acrylate units and monomer selected from styrene, acrylonitrile, methacrylonitrile and methyl methacrylate and use of these compositions for treating the surface of paper, paper products and inkjet papers.

In view of the foregoing, there remains a need for a fragrance delivery system and composition which is (i) capable of delivering fragrance in a consistent manner; (ii) an effective fragrance release system which is stable; and (iii) which lasts effectively for longer duration as desirable.

### SUMMARY OF THE INVENTION

The primary objective of the present application is to provide an oil soluble composition comprising copolymer derived from (i) a first monomer selected from hydrophobically modified (alk)acrylate moiety and (ii) a second monomer having repeating units of an acryloyl and lactam moiety.

One aspect of the present application is to provide an oil soluble composition comprising a copolymer of repeating units of: (i) about 0.1-99.9, preferably 10-90 wt. % of at least one monomer selected from at least one hydrophobically modified (alk)acrylate moiety; and (ii) about 0.1-99.9, preferably 10-90 wt. % of at least one monomer derived from at least one functionalized or unfunctionalized acryloyl moiety and at least one lactam moiety.

According to one specific aspect of the present application, the copolymer is Lauryl(meth)acrylate-hydroxyethylpyrrolidone methacrylate copolymer, and wherein, said “hydroxyethylpyrrolidone methacrylate” is synonymously known to a person skilled in the relevant art as “N-(2-hydroxyethyl) pyrrolidone methacrylate” and has a CAS No: 946-25-8.

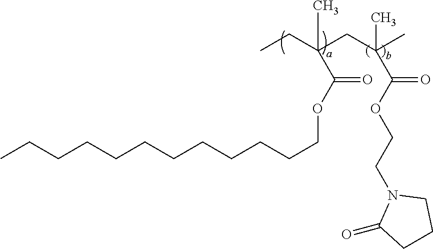

In another aspect, the present application specifically provides a mineral oil soluble personal care composition comprising: (a) about 0.1 to about 99.9 wt. % of a copolymer having repeating units of: (i) from about 0.1-99.9, preferably 10-90 wt. % of at least one monomer selected from at least one hydrophobically modified (alk)acrylate moiety; and (ii) from about 0.1-99.9 preferably 10-90 wt. % of at least one monomer derived from at least one functionalized or unfunctionalized acryloyl moiety and at least one lactam moiety and (b) about 0.1 to about 99.9 wt. % of at least one pharmaceutically or cosmetically acceptable excipient.

In another important aspect, the present application provides a mineral oil soluble fragrance composition comprising: (a) about 0.1 to about 99.9 wt. % of a copolymer having repeating units of: (i) from about 0.1-99.9 preferably 10-90 wt. % of at least one monomer selected from at least one hydrophobically modified (alk)acrylate moiety; and (ii) from about 0.1-99.9 preferably 10-90 wt. % of at least one monomer derived from at least one functionalized or unfunctionalized acryloyl moiety and at least one lactam moiety and (b) about 0.1 to about 99.9 wt. % of at least one pharmaceutically or cosmetically acceptable excipient.

In one another aspect, the present application provides a delivery system for oil soluble functional ingredients comprising: (a) about 0.1 to about 99.9 wt. % of a copolymer having repeating units of: (i) from about 0.1-99.9 preferably 10-90 wt. % of at least one monomer selected from at least one hydrophobically modified (alk)acrylate moiety; and (ii) from about 0.1-99.9 preferably 10-90 wt. % of at least one monomer derived from at least one functionalized or unfunctionalized acryloyl moiety and at least one lactam moiety; and (b) about 0.1 to about 99.9 wt. % of at least one oil soluble functional ingredients.

In yet another aspect, the present application provides a fragrance delivery system for keratin based substrate comprising: (a) about 1 to 25% of fragrance, an active ingredient (b) about 75 to 99% of an emulsion concentrate comprising a copolymer having repeating units derived from: (i) from about 0.1-99 preferably 10-90 wt. % of at least one monomer selected from at least one hydrophobically modified (alk)acrylate moiety; and (ii) from about 0.1-99 preferably 10-90 wt. % of at least one monomer derived from at least one functionalized or unfunctionalized acryloyl moiety and at least one lactam moiety, and (iii) required quantity of water to make up the composition.

According to one important aspect, the desired emulsion for the present application can be a nanoemulsion, microemulsion, or mini emulsion and is combined with a surfactant, or mixture of surfactant systems selected from shampoos, body washes and rinse-off conditioners.

In still another aspect, the present application provides a mineral oil soluble composition comprising the above-described copolymers that enable delivery of fragrance substances gradually and thereby allowing the fragrance to last for at least about 8 hours of duration.

In still another aspect, the present application provides a method of delivering fragrance from a shampoo for hair care comprising (a) about 1 to 25 wt. % of fragrance, an active ingredient (b) about 75 to 99% of an emulsion concentrate including a copolymer having repeating units derived from: (i) from about 0.1-00.9 preferably 10-90 wt. % of at least one monomer selected from at least one hydrophobically modified (alk)acrylate moiety; and (ii) from about 0.1-99.9 preferably 10-90 wt. % of at least one monomer derived from at least one functionalized or unfunctionalized acryloyl moiety and at least one lactam moiety, and (c) required quantity of water.

### BRIEF DESCRIPTION OF THE FIGURES

Further embodiments of the present application can be understood with the appended figures

FIG. 1 depicts long lasting fragrance effects from shampoo compositions of this invention.

FIG. 2 depicts a Panel Study Paired Comparison of a shampoo composition of this invention and a control.

### DETAILED DESCRIPTION OF THE INVENTION

While this specification concludes with claims particularly pointing out and distinctly claiming that, which is regarded as the invention it is anticipated that the invention can be more readily understood through reading the following detailed description of the invention and study of the included examples.

All references to singular characteristics or limitations of the present invention shall include the corresponding plural characteristic or limitation, and vice-versa, unless otherwise specified or clearly implied to the contrary by the context in which the reference is made.

Numerical ranges as used herein are intended to include every number and subset of numbers contained within that range, whether specifically disclosed or not. Further, these numerical ranges should be construed as providing support for a claim directed to any number or subset of numbers in that range.

References herein to “one embodiment,” or “one aspect” or “one version” or “one objective” of the invention may include one or more of such embodiment, aspect, version or objective, unless the context clearly dictates otherwise.

As used herein, the term “alkyl” refers to a functionalized or unfunctionalized monovalent straight-chain, branched-chain or cyclic C₁-C₆₀ group optionally having one or more heteroatoms. Particularly, an alkyl is a C₁-C₄₅ group and more particularly, a C₁-C₃₀ group. Non-limiting examples of alkyl groups include methyl, ethyl, n-propyl, isopropyl, n-butyl, isobutyl, tert-butyl, cyclobutyl, n-pentyl, isopentyl, cyclopentyl, n-hexyl, cyclohexyl, n-heptyl, cyclyheptyl, methylcyclohexyl, n-octyl, 2-ethylhexyl, tert-octyl, iso-norbornyl, n-dodecyl, tert-dodecyl, n-tetradecyl, n-hexadecyl, n-octadecyl, n-eicosyl and the like. The definition of “alkyl” also includes groups obtained by combinations of straight-chain, branche d-chain and/or cyclic structures.

As used herein, the term “(alk) acrylate” refers to an acrylic acid or an alkyl acrylic acid such as methacrylic acid.

As used herein, the term “comprising” refers that various optional, compatible components that can be used in the compositions herein, provided that the important ingredients are present in the suitable form and concentrations. The term “comprising” thus encompasses and includes the more restrictive terms “consisting of” and “consisting essentially of” which can be used to characterize the essential ingredients of the disclosed composition.

As used herein, the term “copolymer” refers to a polymer consisting essentially of two types of repeating structural units (monomers). The definition includes copolymers having solvent adducts.

As used herein, the term “functionalized” refers to the state of a moiety that has one or more functional groups introduced to it by way of one or more functionalization reactions known to a person having ordinary skill in the art. Particularly, functionalization of a moiety replaces one or more functionalizations known to a person having ordinary skill in the art. Yet another non-limiting examples of functionalization reactions include epoxidation, sulfonation, hydrolysis, amidation, esterification, hydroxylation, dihyroxylation, amination, ammonolysis, acylation, nitration, oxidation, dehydration, elimination, hydration, dehydrogenation, hydrogenation, acetalization, halogenation, dehydrohalogenation, Michael addition, aldol condensation, Canizzaro reaction, Mannich reaction, Clasien condensation, Suzuki coupling, and the like.

As used herein, the term “HEPMA” refers to “hydroxyethyl pyrrolidone methacrylate” or N-(2-hydroxyethyl) pyrrolidone methacrylate or hydroxyethylpyrolidone methacrylate or Pyrrolidonylethyl methacrylate (PyEMA) and it has synonymously used in this application, the structure the same (CAS NO: 946-25-8) is provided below:

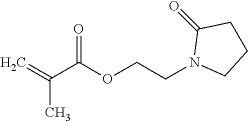

As used herein, the term “hydrophobe” refers to a monomer having solubility in water of less than about 1 percent by weight at 25° C.

As used herein, the term “hydrophobically modified” refers to a functional group in a monomer or copolymer or polymer being replaced by a hydrophobe.

As used herein, the term “keratin substrate” refers to human keratinous surface, and includes skin, nails, and “kerain fibers”, and wherein the “keratin fibers” means hair on head, eyelashes, and eyebrows and other mammalian bodily hair.

As used herein, the term “LMA” refers to “lauryl methyl acrylate”.

As used herein, the term “moiety” refers to a part or a functional group of a molecule.

As used herein, the term “oil” refers to any oil or any solvent that facilitates or enables solubilization of desired active ingredient, preferably a fragrance, and having a Log P or octanol/water partitioning coefficient ranging in values between 0.5 to 6.5. Oil sources comprise mineral (petroleum) oil sources and can be a mixture of long chain hydrocarbons with no triglycerides. The solvent can be alcohols, terpenes, nitriles, ethers, amides, esters, ketones, linear or cyclic hydrocarbons and the like.

As used herein, the term “polymer” refers to a compound comprising repeating structural units (monomers) connected by covalent bonds. The definition includes oligomers. Polymers can be further derivatized (example by hydrolysis), crosslinked, grafted or end-capped. Non-limiting examples of polymers include copolymers, terpolymers, quaternary polymers, and homologues. A polymer may be a random, block, or an alternating polymer, or a polymer with a mixed random, block, and/or alternating structure. Polymers may further be associated with solvent adducts.

As used herein, the term “poly dispersity index” or “PDI” refers to measure of heterogeneity in sizes of molecules or particles in a mixture and refers to either the molecular mass of degree of polymerization.

As used herein, the term “perfumes and fragrances” typically comprise components which react with human olfactory sites resulting in what is known as “fragrance.” Typical molecules which comprise perfume fragrances are linear and cyclic alkenes, i.e., terpenes, primary, secondary and tertiary alcohols, nitrites, ethers, saturated and unsaturated aldehydes, esters, ketones, and mixtures thereof.

As used herein, the terms “personal care composition” and “cosmetics” refer to compositions intended for use on human body, such as skin, sun, hair, oral, cosmetic, and preservative compositions, including those to alter the color and appearance of the skin and hair.

As used herein, the phrase “pharmaceutically acceptable” or “cosmetically acceptable” refers to molecular entities and compositions that are generally regarded as safe. Particularly, as used herein, the term “pharmaceutically acceptable” or “cosmetically acceptable” means approved by a regulatory agency of the appropriate governmental agency or listed in the U.S. Pharmacopoeia or other generally recognized pharmacopoeia for use in animals, and more particularly in humans.

All percentages, ratio, and proportions used herein are based on a weight basis unless otherwise specified.

What is described herein is an oil soluble composition comprising a copolymer having repeating units of: (i) about 10-90 wt. % of at least one monomer selected from at least one hydrophobically modified (alk)acrylate moiety; and (ii) about 30-70 wt. % of at least one monomer derived from at least one functionalized or unfunctionalized acryloyl moiety and at least one lactam moiety. Further described are applications of said oil soluble compositions for thickening oil based functional ingredients such as fragrances.

According to a non-limiting embodiment, the present application provides a mineral oil soluble composition comprising a copolymer of repeating units derived from: (i) from about 10-90 wt. % of at least one monomer selected from at least one hydrophobically modified (alk)acrylate moiety; and (ii) from about 30-70 wt. % of at least one monomer derived from at least one functionalized or unfunctionalized acryloyl moiety and at least one lactam moiety.

According to another non-limiting embodiment, the hydrophobically modified (alk)acrylate moiety refers to an acrylate compound containing a hydrophobe. The hydrophobe can be aliphatic, cycloaliphatic, aromatic or heterocyclic alkyl groups that are straight or long chain having a carbon chain length of C₄-C₃₀ carbons which are functionalized or unfunctionalized.

Examples of suitable hydrophobically modified (alk)acrylate would include, but are not limited to: (meth)acrylic acid or (meth)acrylates encompass: long- and short-chain alkyl (meth)acrylates such as methyl (meth)acrylate, ethyl (meth)acrylate, propyl (meth)acrylate, isopropyl (meth)acrylate, butyl (meth)acrylate, amyl (meth)acrylate, isobutyl (meth)acrylate, t-butyl (meth)acrylate, pentyl (meth)acrylate, isoamyl (meth)acrylate, hexyl (meth)acrylate, heptyl (meth)acrylate, octyl (meth)acrylate, isooctyl (meth)acrylate, 2-ethylhexyl (meth)acrylate, nonyl (meth)acrylate, decyl (meth)acrylate, isodecyl (meth)acrylate, undecyl (meth)acrylate, dodecyl (meth)acrylate, lauryl (meth)acrylate, octadecyl (meth)acrylate, and stearyl (meth)acrylate; alkoxyalkyl (meth)acrylates, particularly d-alkoxy d-alkyl (meth)acrylates, such as butoxyethyl acrylate and ethoxyethoxyethyl acrylate; aryloxyalkyl (eth)acrylate, particularly aryloxy C₄ alkyl (meth)acrylates, such as phenoxyethyl acrylate (e.g., Ageflex, Ciba Specialty Chemicals) single and multi-ring cyclic aromatic or non-aromatic acrylates such as cyclohexyl acrylate, benzyl acrylate, dicyclopentadiene acrylate, dicyclopentanyl acrylate, tricyclodecanyl acrylate, bornyl acrylate, isobornyl acrylate (e.g., Ageflex IBOA, Ciba Specialty Chemicals), tetrahydrofurfuryl acrylate (e.g., SR285, Sartomer Company, Inc.), caprolactone acrylate (e.g., SR495, Sartomer Company, Inc.), and acryloylmorpholine; alcohol-based (meth)acrylates such as polyethylene glycol monoacrylate, polypropylene glycol monoacrylate, methoxyethylene glycol acrylate, methoxypolypropylene glycol acrylate, methoxypolyethylene glycol acrylate, ethoxydiethylene glycol acrylate, and various alkoxylated alkylphenol acrylates such as ethoxylated (4) nonylphenol acrylate (e.g., Photomer4003, Henkel Corp.); amides of (meth)acrylic acid such as diacetone acrylamide, isobutoxymethyl acrylamide, and t-octyl acrylamide; and esters of polyfunctional unsaturated acids, such as maleic acid ester and fumaric acid ester. Preferred examples of hydrophobically modified (alk)acrylate include lauryl (meth)acrylate, ethylhexyl (meth)acrylate and stearyl (meth)acrylate.

According to one important embodiment of the present application, the copolymer comprises a second monomer derived from at least one functionalized or unfunctionalized acryloyl moiety and at least one lactam moiety having a structure:

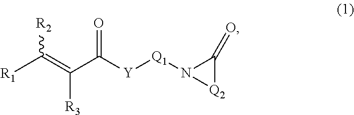

wherein each R₁ R₂ and R₃ is independently selected from the group derived from hydrogen, halogens, functionalized and unfunctionalized C₁-C₄ alkyl, and

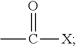

each X is independently selected from the group derived from OR₄, OM, halogen, N(R₅)(R₆),

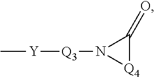

and combinations thereof; each Y is independently oxygen, NR₇ or sulfur; each R₄, R₅, R₆ and R₇ is independently selected from the group derived from hydrogen and functionalized and unfunctionalized alkyl; each M is independently selected from the group derived from metal ions, ammonium ions, organic ammonium cations, and combinations thereof; and each Q₁, Q₂, Q₃, and Q₄ is independently selected from the group derived from functionalized and unfunctionalized alkylene.

Particularly, each Q1, Q2, Q3, and Q4 is independently selected from the group derived from functionalized and unfunctionalized C1-C12 alkylene. In Particular, yet non-limiting examples of alkylene groups include —CH2-. —CH2-CH2-, —CH(CH3)-CH2-, —CH2-CH(CH3)-, —C(CH3)2-CH2-, —CH2-C(CH3)2-, —CH(CH3)-CH(CH3)-, —C(CH3)2-C(CH3)2-, —CH2-CH2-CH2-, —CH(CH3)-CH2-CH2-, —CH2-CH(CH3)-CH2-, —CH2-CH2-CH(CH3)-, —CH2-CH2-CH2-CH2-, —CH2-CH2-CH2-CH2-CH2-, —CH2-CH2-CH2-CH2-CH2-CH2-, and —CH2-CH2-CH2-CH2-CH2-CH2-CH2-.

In one non-limiting embodiment, each R₁, R₂ and R₃ is independently selected from the group consisting of hydrogen, methyl and combinations thereof. Particularly, R₁ and R₂ are hydrogen and R₃ is hydrogen or methyl.

In another non-limiting embodiment, each R₁ and R₃ is independently hydrogen or methyl; R₂ is

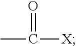

X is selected from the group derived from OR₄, OM, halogens, and N(R₅)(R₆); wherein each R₄, R₅, and R₆ is independently selected from the group consisting of hydrogen and functionalized and unfunctionalized alkyl; and each M is independently selected from the group consisting of metal ions, ammonium ions, organic ammonium cations, and combinations thereof.

Particularly, R₁ and R₃ are hydrogens and R₂ is

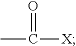

X is selected from the group consisting of OR₄, OM and N(R₅)(R₆); each R₄, R₅, and R₆ is independently selected from the group consisting of hydrogen and functionalized and unfunctionalized C₁-C₄ alkyl; and each M is independently selected from the group consisting of metal ions, ammonium ions, organic ammonium cations, and combinations thereof.

The first polymerizable unit, defined by structure (1), can be synthesized using methods recorded in the art, e.g., by reaction of an N-hydroxylalkyl lactam with an acrylate, (meth)acrylate, anhydride, or similar compounds. Production methods include those described in U.S. Pat. Nos. 2,882,262; 5,523,340; 6,369,163; U.S. Patent Application Publication No. 2007/123673; GB Patent No.'s 924,623; 930,668; and 1,404,989; PCT Publication No. WO 03/006569; and EP Patent No. 385918. Each of the previous disclosures are hereby incorporated herein by reference in its entirety.

The lactam moiety containing monomers shown in structures (2)-(57) can be obtained from condensation reactions that include an N-hydroxyalkyl lactam and an unsaturated carboxylic acid, an acrylate, a (meth)acrylate, or an anhydride. Suitable N-hydroxyalkyl lactams include N-hydroxymethyl pyrrolidone and caprolactam, N-hydroxyethyl pyrrolidone and caprolactam, and N-hydroxypropyl pyrrolidone and caprolactam. Non-limiting examples of carboxylic acids that can be used include: acrylic acid, methacrylic acid, itaconic acid, crotonic acid, fumaric acid, succinic acid, and maleic acid. Similarly, acrylates and (meth)acrylates include (without limitation) methyl, ethyl, butyl, octyl, ethyl hexyl acrylates and their (meth)acrylate analogues. Representative anhydrides include formic anhydride, succinic anhydride, maleic anhydride and acetic anhydride.

In particular embodiments, the monomer having at least one functionalized or unfunctionalized acryloyl moiety and at least one lactam moiety has a structure selected from the group consisting of:

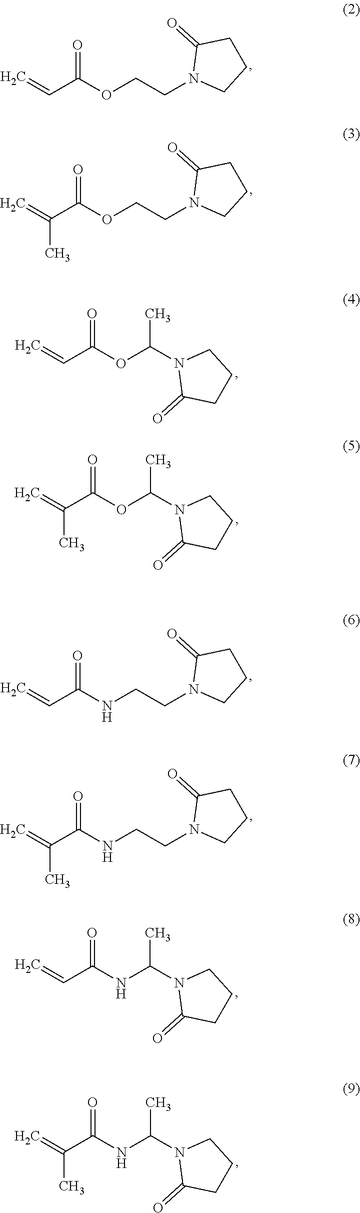

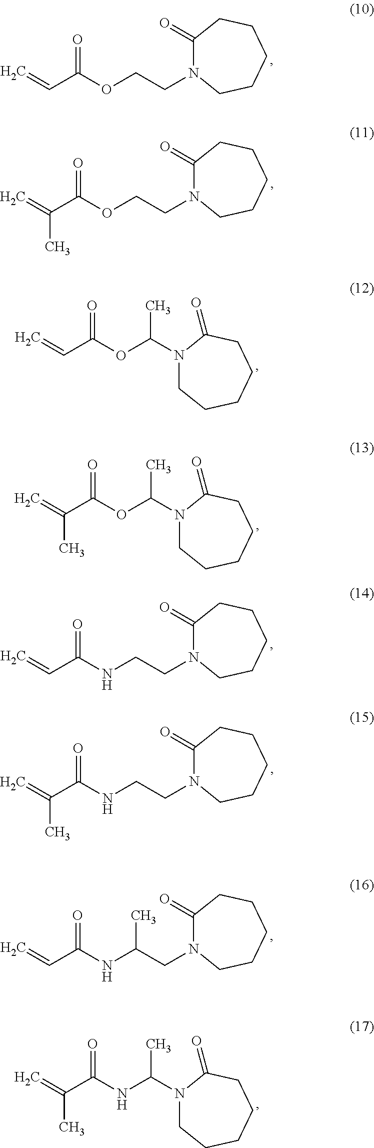

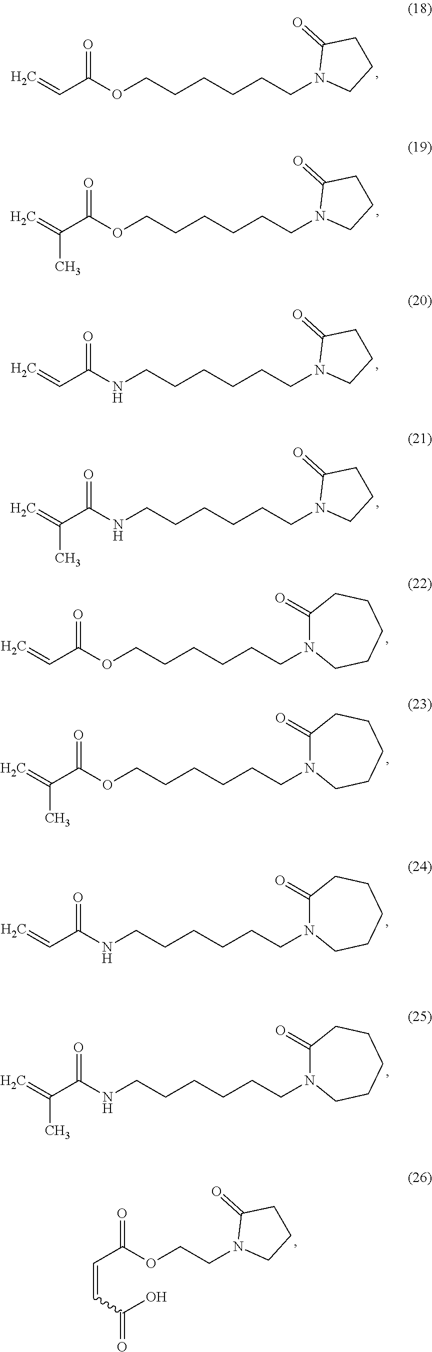

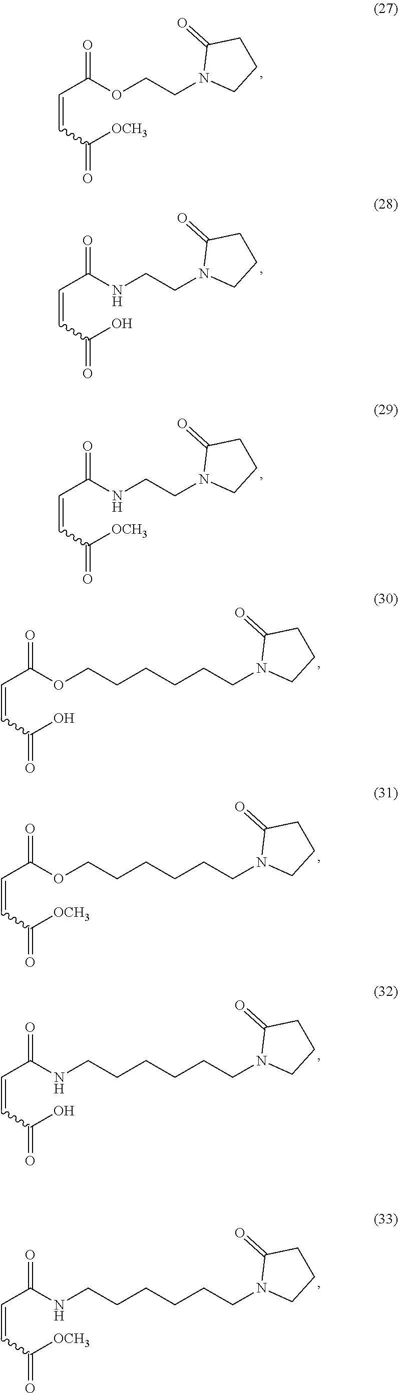

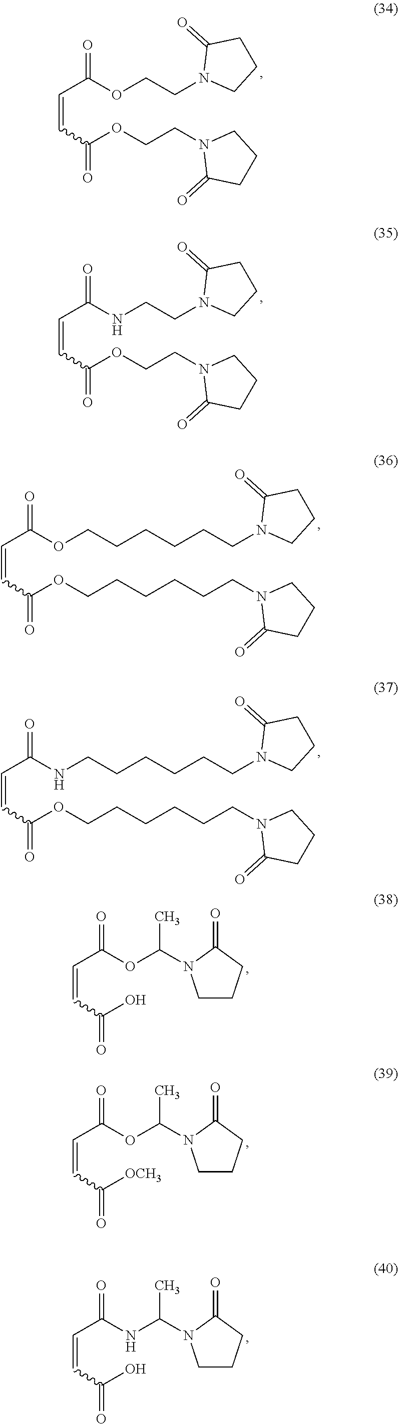

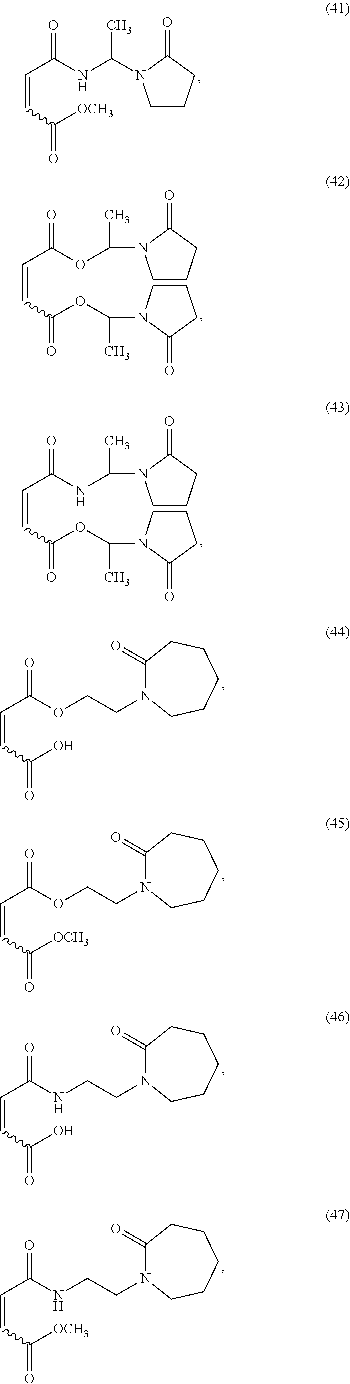

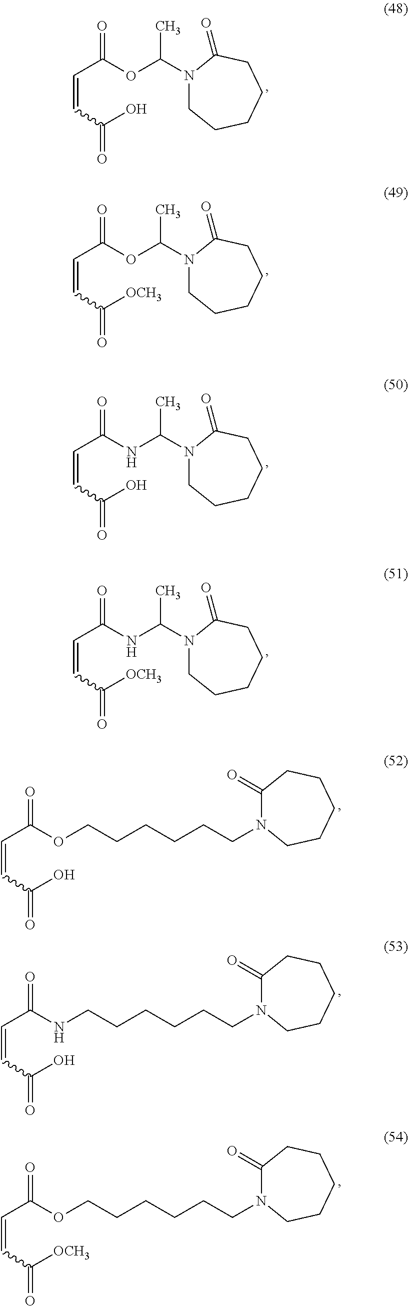

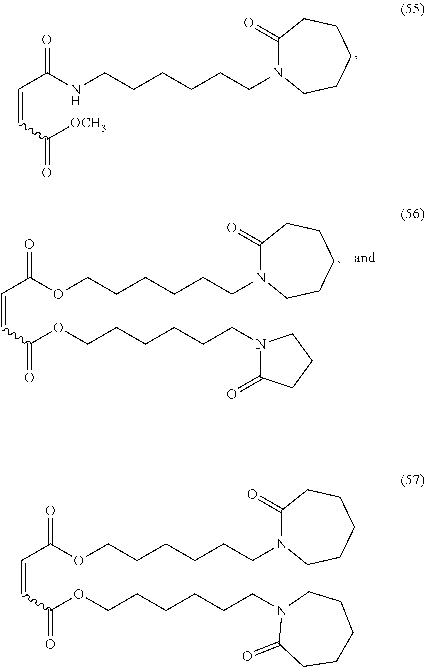 [SMILES: COC(=O)C=CC(=O)NCCCCCCN1CCCCCC1=O ; O=C(C=CC(=O)OCCCCCCN1CCCC1=O)OCCCCCCN1CCCCCC1=O ; O=C(C=CC(=O)OCCCCCCN1CCCCCC1=O)OCCCCCCN1CCCCCC1=O]

Other suitable examples relating to functionalized or unfunctionalized acryloyl moiety and at least one lactam moiety can be found in PCT Publication No. WO2011/063208, the disclosure of which is incorporated herein by reference in its entirety.

According to another important embodiment of the present application, the copolymer has repeating units of: (i) from about 40 to about 80 percent by weight of at least one first monomer is selected from the group consisting of 2-ethylhexyl acrylate, 2-ethylhexyl methacrylate, N-2-ethylhexyl acrylamide, N-2-ethylhexyl methacrylamide, and combinations thereof; and (ii) from about 20 to about 60 percent by weight of at least one second monomer having a structure selected from the group derived from the following structures (a1)-(a8):

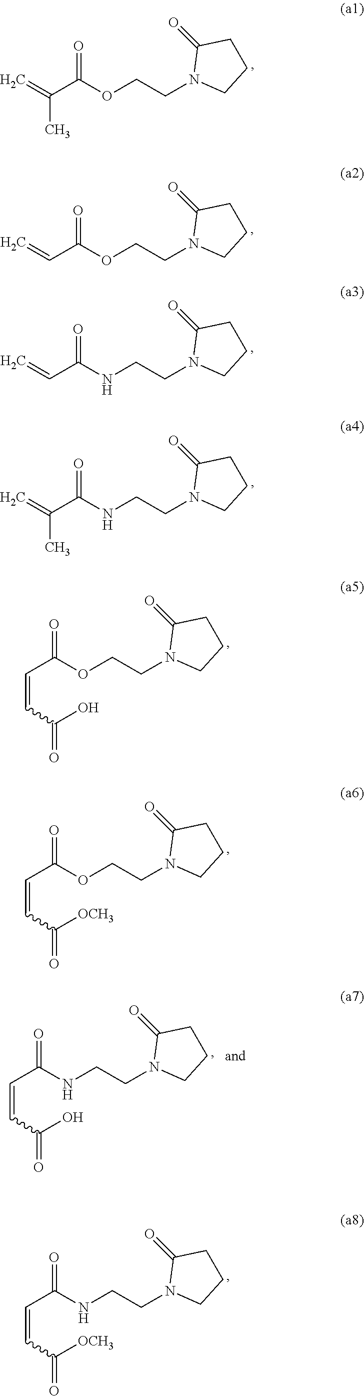

and combinations thereof.

According to another important embodiment of the present application, the copolymer of the present application has a structure selected from the group consisting of:

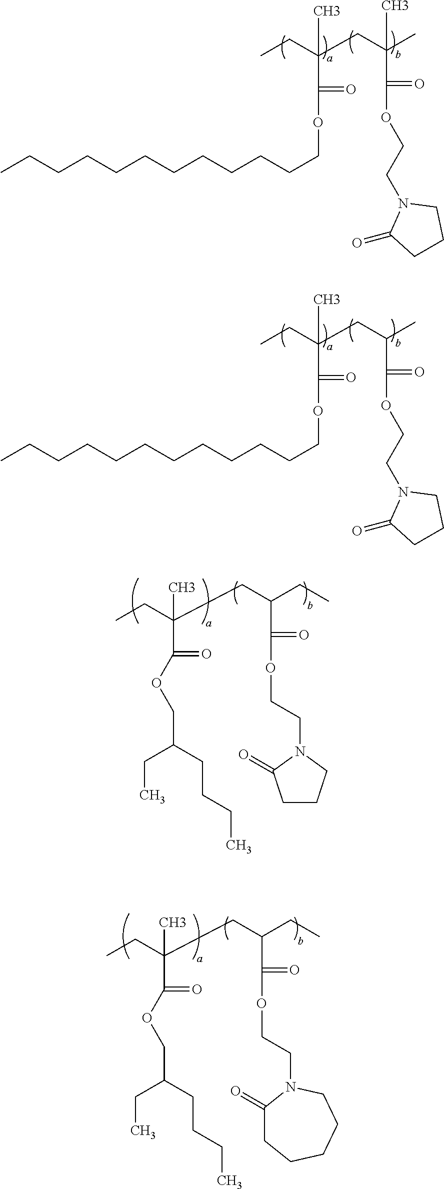

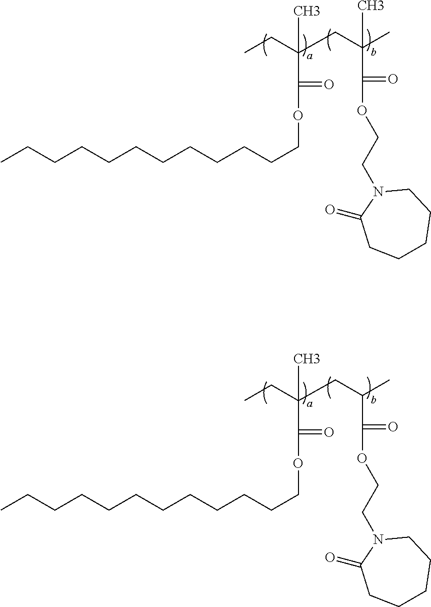

wherein each a, and b is an independently selected value ranging from about 0.1 to about 99.9 percent by weight of the polymer, with the proviso that the sum of a and b for each polymer equals 100 weight percent.

According to another important embodiment of the present application, the copolymer is Lauryl(meth)acrylate-hydroxyethyl pyrrolidone methacrylate copolymer having a structure of:

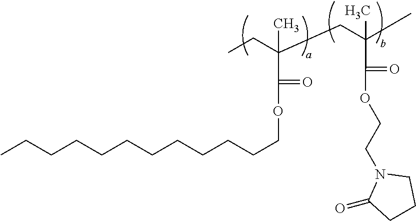

The copolymer is soluble in mineral oil and has an average molecular weight of at least about 10,000 to about 800,000 Daltons. Other preferred ranges of molecular weights are from about 120000 to about 150,000, from about 150,000 to about 200,000, from about 200,000 to about 250000; from about 250,000 to about 300000. Particularly, Lauryl methacrylate used in the present invention has a molecular weight in different ranges selected from about 600,000 to about 650,000; from about 700,000 to about 750,000; from about 350,000 to about 400,000, from about 500,000 to 550,000; from about 200,000 to about 240,000; from about 150,000 to about 160,000; from about 70,000 to 75,000; from about 175,000 to about 185,000; about 200,000; from about 300,000 to about 320,000; around 20,000; and from about 500,000 to about 550,000. The monomers Lauryl (meth)acrylate and hydroxyethyl pyrrolidone methacrylate are present in different weight ratios of about 90/10, 10/90, 25/75, 75/25, 80/20, 20/80, 60/40, 40/60 and 50/50 respectively.

The specific monomers optionally employed as the at least one crosslinking agent to prepare desired repeating units or to prepare copolymer of the present application, can be selected from divinyl ethers of compounds selected from the group derived from ethylene glycol, 1,3-propanediol, 1,4-butanediol, 1,5-pentanediol, 1,6-hexanediol, 1,7-heptanediol, 1,8-octanediol, 1,9-nonanediol, 1,10-decanediol, 1,11-unidecanediol, 1,12-dodecanediol, and combinations thereof; divinyl ethers of diethylene glycol, triethylene glycol, tetraethylene glycol, pentaethylene glycol, hexaethylene glycol, heptaethylene glycol, octaethylene glycol, nonaethylene glycol, decaethylene glycol, and polyalkylene glycols; methylenebis(meth)acrylamide; ethylene glycol di(meth)acrylate; butanediol di(meth)acrylate; tetraethylene glycol di(meth)acrylate; polyethylene glycol di(meth)acrylate; dipropylene glycol diallyl ether; polyglycol diallyl ether; hydroquinone diallyl ether; trimethylolpropane tri(meth)acrylate; trimethylolpropane diallyl ether; pentaerythritol triallyl ether; allyl(meth)acrylate; triallyl cyanurate; diallyl maleate; polyallyl esters; tetraallyloxyethane; triallylamine; tetraallylethylenediamine; divinyl benzene; glycidyl (meth)acrylate; 1,7-octadiene; 1,9-decadiene; 1,13-tetradecadiene; divinylbenzene; diallyl phthalate; triallyl-1,3,5-triazine-2,4,6(1H,3H,5H)-trione; N,N′-divinylimidazolidone; 1-vinyl-3(E)-ethylidene pyrrolidone; 2,4,6-triallyloxy-1,3,5-triazine; and combinations thereof.

According to one embodiment of the present application, the compositions can be used as such or formulated with other ingredient(s) to result in desired type of product forms.

According to one important embodiment of the present application, the suitable oil employed for solubilizing the composition of the present application is specifically chosen from any oil or a solvent having a Log P value or octanol/water partition coefficient of about 0.5 to about 6.5, and wherein, Log P values refers to the lipophilicity of the unionized species and lipophilicity changes as a function of pH for ionisable compounds. Non-limiting examples of oils or solvents having Log P value from 0.5 to 6.5 comprise mineral oil (4.7 to 6), diethyl ether (0.83), p-dichlorobenzene (4.61), 2,2′,4,4′-pentachlorophenyl (6.41), phenoxyethanol (1.08-1.1), peppermint oil (2.9 to 3.2), spearmint oil (2.5 to 3.0), Eucalyptol (3.22), Limonene laevo (4.46), Menthol laevo (3.20), Menthone (2.63), Menthyl acetate (4.10), Pinene Alpha (4.46), Pinene Beta (4.37), Alcohol CIO (decanol) (4.06), Anethole (3.17), Eugenol (2.79), Hexyl Cinnamic Aldehyde (4.67), Ionone beta (3.91), Methyl salicylate (2.40), Terpinyl propionate (4.245), Isododecane (Log P 6.16), isododecane, Hydroxycitronellal (4.216), Hydrogenated Didecene, Hydrogenated Didodecene, Hydrogenated Polydecene, Hydrogenated Polydodecene, Hydrogenated Tridodecene, Hydrogenated Polyisobutene, and a mixture of two or more thereof. Solvents having a Log P values from 0.7 to 1.3 include benzyl alcohol, cis-3-hexenol, phenyl ethyl alcohol, methylbenzyl alcohol, anisyl alcohol, isoamyl alcohol, 4-hexen-1-ol, phenoxyethanol, phenoxypropanediol, trimethyl-1,3-pentanediol, chlorphenesin, ethylhexyl glycerin, caprylyl glycol, glyceryl caprylate, hexanediol, 1,2-hexane diol, ethyl hexanediol, pentylene glycol, octanediol, hydroxypropyl methacrylate, triethyl citrate or mixtures thereof. Also contemplated are silicones selected from linear silicones of the dimethicone type, cyclic silicones or a mixture thereof.

According to another important embodiment of the present application, the oil soluble composition is a personal care composition, cementing fluid composition, oilfield composition, construction composition, servicing fluid composition, gravel packing mud composition, fracturing fluid composition, completion fluid composition, workover fluid composition, spacer fluid composition, drilling mud composition, coating composition composition, household composition, industrial and institutional composition, pharmaceutical composition, food composition, biocide composition, adhesive composition, ink composition, paper composition, polish composition, membrane composition, metal working fluid composition, plastic composition, textile composition, printing composition, lubricant composition, preservative composition, agrochemical composition, or wood-care composition. Particularly, the non-aqueous composition is a personal care composition, coating composition, household composition, industrial and institutional composition, pharmaceutical composition, or an agricultural composition. More particularly, the non-aqueous composition is a personal care composition.

According to another important embodiment of the present application, the mineral oil soluble composition is a personal care composition comprising a copolymer having repeating units of: (i) from about 0.1-99.9, preferably 10-90, wt. % of at least one monomer selected from at least one hydrophobically modified (alk)acrylate moiety; and (ii) from about 0.1-99.9, preferably 10-90, wt. % of at least one monomer derived from at least one functionalized or unfunctionalized acryloyl moiety and at least one lactam moiety.

According to one important embodiment of the present application, the mineral oil soluble personal care composition is a sun care composition, a face care composition, a lip care composition, an eye care composition, a skin care composition, an after-sun composition, a body care composition, a nail care composition, an anti-aging composition, an insect repellant composition, an oral care composition, a deodorant composition, a hair care composition, a conditioning composition, a color cosmetic composition, a color-protection composition, a self-tanning composition, a fragrance composition or a foot care composition.

According to another important embodiment of the present application, the composition further comprises at least one pharmaceutically or cosmetically acceptable ingredient. “Cosmetically acceptable ingredient” as used herein means any ingredient/compound or mixture of ingredients/compounds or compositions that are typically employed to produce other desirable effects in personal care compositions. The preferred cosmetically acceptable excipients include, but are not limited to, preservatives, antioxidants, chelating agents, sunscreen agents, proteins, amino acids, vitamins, dyes, hair coloring agents, plant extracts, plant derivatives, plant tissue extracts, plant seed extracts, plant oils, botanicals, botanical extracts, humectants, fragrances, perfumes, oils, emollients, lubricants, butters, penetrants, thickeners, viscosity modifiers, thickeners, polymers, resins, hair fixatives, film formers, surfactants, detergents, emulsifiers, opacifying agents, volatiles, propellants, liquid vehicles, carriers, salts, pH adjusting agents, neutralizing agents, buffers, hair conditioning agents, anti-static agents, anti-frizz agents, anti-dandruff agents, hair waving agents, hair straightening agents, relaxers, absorbents, fatty substances, gelling agents, moisturizers, hydrophilic or lipophilic active agent, preserving agents, fillers, dyestuffs, reducing agents, cosmetic oils, perfumes, liquid vehicles, solvents, carriers, silicones, and combinations thereof. Particularly, cosmetically acceptable ingredients can be selected from the group derived from oils, waxes, triglycerides, fatty esters, fatty amides, fatty hydrocarbons, and combinations thereof.

Conditioning agents can be chosen from synthetic oils, mineral oils, vegetable oils, fluorinated or perfluorinated oils, natural or synthetic waxes, silicones, cationic polymers, proteins and hydrolyzed proteins, cationic surfactants, ceramide type compounds, fatty amines, fatty acids and their derivatives, as well as mixtures of these different types of compounds.

Surfactant based conditioning agents include cocamidopropyl betaine, coconut oil, hydrolyzed animal protein, keratin, collagen and the like.

Suitable non-limiting examples of cationic polymers include quaternary cationic polymers selected from acrylamidopropyl trimonium chloride (APTAC), polyquaternium-22, polyAPTAC, Guar hydroxypropyltrimonium chloride, diallyldimethylammonium chloride/acrylic acid copolymer, APTAC/acrylamide copolymer and the like.

Suitable non-limiting examples of anionic surfactants include sulfate, sulfonate, carboxylate anion based surfactant, ether sulfate, ethoxy sulfate, propoxy sulfate, C₃₂H₆₅O—PO₇-EO6-SO₃—, C₁₂₋₁₅-3EO sulfate, C₁₂₋₁₅-12EO sulfate, C₁₆₋₁₇-7PO sulfate, C₁₃-7PO sulfate, C₁₆₋₁₈-7PO-5EO sulfate, C₂₀-7PO-10EO sulfate, perfluorooctanoate (PFOA or PFO), perfluorooctanesulfonate (PFOS), sodium dodecyl sulfate (SDS), ammonium lauryl sulfate, alkyl sulfate salt, sodium lauryl ether sulfate (SLES), alkyl benzene sulfonate, soap, fatty acid salt, or a combination thereof.

Suitable non-limiting examples of preservatives include sodium chloride, potassium chloride, calcium chloride, ascorbic acid, citric acid, potassium sorbate and the like.

According to another important embodiment of the present application, a delivery system for oil based functional ingredients is provided comprising (a) about 0.1 to about 99.9 wt % of a copolymer having repeating units of (i) from about 0.1-99.9, preferably 10-90, wt. % of at least one monomer selected from at least one hydrophobically modified (alk)acrylate moiety; and (ii) from about 0.1-99.9, preferably 10-90, wt. % of at least one monomer derived from at least one functionalized or unfunctionalized acryloyl moiety and at least one lactam moiety; and (b) about 0.1 to about 99.9 wt. % of at least one oil soluble functional ingredients.

Accordingly, the functional ingredient is preferably a fragrance component that can advantageously be incorporated in the following non-limiting personal care compositions such as shampoos, bath gels, air fresheners, candles, reactive hair care compositions selected from hair dyes, hair bleaches, and odor neturalizers or malodor counteractants.

According to another important embodiment of the present application, there is provided a fragrance delivery system for keratin based substrate such as skin or hair comprising: (a) about 1 to 25 wt. % of fragrance, an active ingredient; (b) about 75 to 99 wt. % of an emulsion concentrate including a copolymer having a repeating units of: (i) from about 0.1-99.9, preferably 10-90, wt. % of at least one monomer selected from at least one hydrophobically modified (alk)acrylate moiety; and (ii) from about 0.1-99.9, preferably 10-90, wt. % of at least one monomer derived from at least one functionalized or unfunctionalized acryloyl moiety and at least one lactam moiety; and (c) required quantity of water.

Accordingly, the delivery system comprises delivery of an active ingredient, preferably a fragrance component onto a keratin based substrates such as hair, skin or nails. The ideal objective of any fragrance delivery system is to provide a consistent release of the fragrance over the scheduled lifetime of the article, and wherein, such consistent release is referred to as “linear release” and is employed to describe a concept whereby a consumer perceives the emitted scent from an article to be identical with respect to quality and intensity throughout the prescribed lifetime of an article. In practicality, this ideal is difficult to achieve because aromachemicals of the fragrance composition have differing vapor pressures and differing threshold values at which a person perceives the odor of the aromachemical. Additionally, and important to the design of the aroma release engine system, consumers have differing abilities to detect the various aromachemicals, and this issue is compounded by effects of differing environmental conditions, e.g., temperature, air flow, humidity, volume of emission space.

Fragrances for the present application can be one or more selected from essential oils, plant extracts, absolutes, resinoids, resins, concretes, hydrocarbons, alcohols, aldehydes, ketones, ethers, esters, acetals, ketals, nitriles, including saturated and unsaturated compounds and aliphatic, carboxylic and heterocyclic compounds. Suitable fragrances include but are not limited to fruits such as almond, apple, cherry, grape, pear, pineapple, orange, strawberry, raspberry; musk, flower scents such as lavender-like, rose-like, iris-like, carnation-like; herbal scents such as rosemary, thyme, and sage; woodland scents derived from pine, spruce and other forest smells; oils such as essential oils, or from plant materials such as peppermint, spearmint, and other familiar and popular smells such as baby powder, popcorn, pizza, cotton candy and the like in the present invention. Natural extracts of fragrances include acacia, cassie, chypre, cylamen, fern, gardenia, hawthorn, heliotrope, honeysuckle, hyacinth, jasmine, lilac, lily, magnolia, mimosa, narcissus, freshly-cut hay, orange blossom, orchids, reseda, sweet pea, trefle, tuberose, vanilla, violet, wallflower, and the like.

The most common product forms of surfactant systems for personal care are shampoos and bath gels. The fragrance can be considered an additive, and its effect on the critical micelle concentration (CMC) will determine the consequences for the viscosity. The shape of the micelle can be determined by a relationship between three parameters: the volume occupied by the hydrophobic groups in the micellar core, the length of the hydrophobic group in the core, and the area occupied by the hydrophilic group at the micelle surface. The individual aroma chemicals in a fragrance will partition into different areas of the surfactant system. Some materials will migrate into the core of the micelle, some will align along the hydrophobic tails, some will be near the micelle surface, and a small amount will be in the external aqueous phase. The number, shape, and size of the micelles, and any thickening that has been created in the external phase, determine the viscosity of the system. The fragrance materials can change any of these parameters, and thus make the viscosity increase or decrease.

Without wishing to be bound by any particular theory, it is believed that the polymer of the present application comprising lauryl methacrylate and hyroxyethylpyrrolidone methacrylate binds with fragrance and does not reside in micelles. Lauryl (metha)acrylate and ethylhexyl methacrylate are stable on hair. Hence this polymer/fragrance complex allows gradual release of fragrance that lasts for at least about 8 hours of duration. Fragrance can be delivered with or without any cationic or amphoteric polymers. However, the presence of a cationic or amphoric copolymer is preferred. Copolymers of the present application are capable of providing thickened composition comprising fragrance and in surfactant systems fragrances partition preferentially in polymer phase. In combination with cationic or amphotenic amphroteric polymers, the copolymers of the present application are capable of providing high levels of deposition. Further, such copolymers do not suppress fragrance component's vapor pressure and hence fragrance lasts longer duration as desired, and moreover, the polymer/surfactant phase does not reduce fragrance vapor pressure totally and thus leads to a gradual release of the deposited or adsorbed fragrance onto a keratin substrate. Suitable cationic or amphoteric polymers include (DMAPMA/APTAC/MA), (APTAC/acrylamide) and (DMAPMA/MA) (dimethyaminopropylmethacrylic acid/methacrylic acid), present in an amount of 0.1-5, preferably 0.5-2% by weight.

In one embodiment, the fragrance delivery system of the present application can be in the form of a nano emulsion, micro emulsion or mini emulsion when combined with surfactant systems that are pertinent to shampoos, body washes, and rinse off conditioners.

The oil soluble composition comprising Lauryl (meth)acrylate-hydroxy methyl pyrrolidone acrylate copolymer when used in shampoo based compositions effectively binds to a fragrance ingredient of the shampoo and releases fragrance gradually and thereby allowing the fragrance to last longer than at least 8 hours.

Yet another important embodiment of the present application provides a method of delivering fragrance from a shampoo for hair care comprises: (a) from about 1 to about 25 wt. % of fragrance, an active ingredient, (b) 75 to 99 wt. % of an emulsion concentrate comprising a copolymer having repeating units of: (i) from about 0.1 to about 99.9, preferably 10-90, wt. % of at least one monomer selected from at least one hydrophobically modified (alk)acrylate moiety; and (ii) from about 0.1-99.9, preferably 10-90, wt. % of at least one monomer derived from at least one functionalized or unfunctionalized acryloyl moiety and at least one lactam moiety; and (c) required quantity of water.

Further, certain aspects of the present invention are illustrated in detail by way of the following examples. The examples are given herein for illustration of certain aspects of the invention and are not intended to be limiting thereof.

### EXAMPLES

### Examples 1-7: Lauryl Methacrylate (80 wt. %)/Hydroxyethyl Pyrrolidone Methacrylate (20 wt. %) Co-Polymer

A heel comprising Lauryl Methacrylate (12 g), Hydroxyethyl-Pyrrolidone Methacrylate (3 g) in 100 g t-butanol was added to reactor and purged three times with nitrogen to deoxygenate the system. This heel solution was then heated to 80° C., one portion of initiator was added, and the feeds of lauryl methacrylate and Hydroxyethyl-Pyrrolidone Methacrylate were commenced. Lauryl methacrylate (108 g) was fed over an hour, while Hydroxyethyl-Pyrrolidone Methacrylate (27 g) combined with (50 g) t-butanol was fed over 2 hours. Every hour a fresh portion of initiator was added. Upon completion of the feeds, the reaction was held for four more hours with additional shots of initiator added hourly. The reaction was then heated to 90° C. and held for 12 hours. The resultant polymer solution was cooled to 35° C. and discharged from the reactor. A viscous water-white solution of polymer is obtained. Polymer solution is solvent exchanged by stripping t-butanol reaction solvent and replacing with applications friendly delivery solvent, most preferably isohexadecane.

### Example 8: Lauryl Methacrylate (60 wt. %)/Hydroxyethyl Pyrrolidone Methacrylate Co-Polymer (40 wt. %)

A heel comprising Lauryl Methacrylate (9 g), Hydroxyethyl-Pyrrolidone Methacrylate (6 g) in 100 g t-butanol was added to reactor and purged three times with nitrogen to deoxygenate the system. This heel solution was then heated to 80° C., one portion of initiator was added, and the feeds of lauryl methacrylate and Hydroxyethyl-Pyrrolidone Methacrylate were commenced. Lauryl methacrylate (81 g) was fed over an hour, while Hydroxyethyl-Pyrrolidone Methacrylate (54 g) combined with (50 g) t-butanol was fed over 2 hours. Every hour a fresh portion of initiator was added. Upon completion of the feeds, the reaction was held for four more hours with additional shots of initiator added hourly. The reaction was then heated to 90° C. and held for 12 hours. The resultant polymer solution was cooled to 35° C. and discharged from the reactor. A viscous water-white solution of polymer is obtained. Polymer solution is solvent exchanged by stripping t-butanol reaction solvent and replacing with applications friendly delivery solvent, most preferably isohexadecane.

### Example 9: Lauryl Methacrylate (40 wt. %)/Hydroxyethyl Pyrrolidone Methacrylate Co-Polymer (60 wt. %)

A heel comprising Lauryl Methacrylate (6 g), Hydroxyethyl-Pyrrolidone Methacrylate (9 g) in 100 g t-butanol was added to reactor and purged three times with nitrogen to deoxygenate the system. This heel solution was then heated to 80° C., one portion of initiator was added, and the feeds of lauryl methacrylate and Hydroxyethyl-Pyrrolidone Methacrylate were commenced. Lauryl methacrylate (54 g) was fed over an hour, while Hydroxyethyl-Pyrrolidone Methacrylate (81 g) combined with (50 g) t-butanol was fed over 2 hours. Every hour a fresh portion of initiator was added. Upon completion of the feeds, the reaction was held for four more hours with additional shots of initiator added hourly. The reaction was then heated to 90° C. and held for 12 hours. The resultant polymer solution was cooled to 35° C. and discharged from the reactor. A viscous water-white solution of polymer is obtained. Polymer solution is solvent exchanged by stripping t-butanol reaction solvent and replacing with applications friendly delivery solvent, most preferably isohexadecane.

### Example 10: Lauryl Methacrylate (90 wt. %)/Hydroxyethyl Pyrrolidone Methacrylate Co-Polymer (10 wt. %)

A heel comprising Lauryl Methacrylate (13.5 g), Hydroxyethyl-Pyrrolidone Methacrylate (1.5 g) in 125 g t-butanol was added to reactor and purged three times with nitrogen to deoxygenate the system. This heel solution was then heated to 80° C., one portion of initiator was added, and the feeds of lauryl methacrylate and Hydroxyethyl-Pyrrolidone Methacrylate were commenced. Lauryl methacrylate (121.5 g) was fed over an hour, while Hydroxyethyl-Pyrrolidone Methacrylate (13.5 g) combined with (50 g) t-butanol was fed over 2 hours. Every hour a fresh portion of initiator was added. Upon completion of the feeds, the reaction was held for five more hours with additional shots of initiator added hourly. The reaction was then heated to 90° C. and held for 12 hours. The resultant polymer solution was cooled to 35° C. and discharged from the reactor. A viscous water-white solution of polymer is obtained.

### Example 11: Lauryl Methacrylate (10 wt. %)/Hydroxyethyl Pyrrolidone Methacrylate Co-Polymer (90 wt. %)

A heel comprising Lauryl Methacrylate (2.25 g), Hydroxyethyl-Pyrrolidone Methacrylate (13.5 g) in 125 g t-butanol was added to reactor and purged three times with nitrogen to deoxygenate the system. This heel solution was then heated to 80° C., one portion of initiator was added, and the feeds of lauryl methacrylate and Hydroxyethyl-Pyrrolidone Methacrylate were commenced. Lauryl methacrylate (12.75 g) was fed over an hour, while Hydroxyethyl-Pyrrolidone Methacrylate (121.5 g) combined with (50 g) t-butanol was fed over 2 hours. Every hour a fresh portion of initiator was added. Upon completion of the feeds, the reaction was held for five more hours with additional shots of initiator added hourly. The reaction was then heated to 90° C. and held for 12 hours. The resultant polymer solution was cooled to 35° C. and discharged from the reactor. A viscous water-white solution of polymer is obtained.

### (Comparative Example A): Lauryl Methacrylate Homopolymer (100 wt. %

A heel comprising Lauryl Methacrylate (20 g) in 100 g t-butanol was added to reactor and purged three times with nitrogen to deoxygenate the system. This heel solution was then heated to 80° C., one portion of initiator was added, and the feed of lauryl methacrylate and remaining t-butanol was commenced. Lauryl methacrylate (130 g) combined with (50 g) t-butanol was fed over 2 hours. Every hour a fresh portion of initiator was added. Upon completion of the feeds, the reaction was held for five more hours with additional shots of initiator added hourly. The reaction was then heated to 90° C. and held for 12 hours. The resultant polymer solution was cooled to 35° C. and discharged from the reactor. A viscous water-white solution of polymer is obtained. Polymer solution is solvent exchanged by stripping t-butanol reaction solvent and replacing with applications friendly delivery solvent, most preferably isohexadecane.

### Example 13: Stearyl Methacrylate (80 wt. %)/Hydroxyethyl Pyrrolidone Methacrylate (20 wt. %)

A heel comprising Stearyl Methacrylate (12 g), Hydroxyethyl-Pyrrolidone Methacrylate (3 g) in 125 g t-butanol was added to reactor and purged three times with nitrogen to deoxygenate the system. This heel solution was then heated to 80° C., one portion of initiator was added, and the feeds of stearyl methacrylate and Hydroxyethyl-Pyrrolidone Methacrylate were commenced. Stearyl methacrylate (108 g) was fed over an hour, while Hydroxyethyl-Pyrrolidone Methacrylate (27 g) combined with (50 g) t-butanol was fed over 2 hours. Every hour a fresh portion of initiator was added. Upon completion of the feeds, the reaction was held for five more hours with additional shots of initiator added hourly. The reaction was then heated to 90° C. and held for 12 hours. The resultant polymer solution was cooled to 35° C. and discharged from the reactor. A viscous water-white solution of polymer is obtained.

### Examples 14-17: Ethylhexyl Methacrylate (80 wt. %)/Hydroxyethyl Pyrrolidone Methacrylate (20 wt. %)

A heel comprising Ethylhexyl methacrylate (12 g), Hydroxyethyl-Pyrrolidone Methacrylate (3 g) in 100 g t-butanol was added to reactor and purged three times with nitrogen to deoxygenate the system. This heel solution was then heated to 80° C., one portion of initiator was added, and the feeds of ethylhexyl methacrylate and Hydroxyethyl-Pyrrolidone Methacrylate were commenced. Ethylhexyl methacrylate (108 g) was fed over an hour, while Hydroxyethyl-Pyrrolidone Methacrylate (27 g) combined with (50 g) t-butanol was fed over 2 hours. Every hour a fresh portion of initiator was added. Upon completion of the feeds, the reaction was held for four more hours with additional shots of initiator added hourly. The reaction was then heated to 90° C. and held for 12 hours. The resultant polymer solution was cooled to 35° C. and discharged from the reactor. A viscous water-white solution of polymer is obtained.

### (Comparative Example B): Lauryl Methacrylate (80 wt. %)/Vinyl Pyrrolidone (20 wt. %)

A heel comprising Lauryl Methacrylate (12 g), Vinyl pyrrolidone (3 g) in 100 g t-butanol was added to reactor and purged three times with nitrogen to deoxygenate the system. This heel solution was then heated to 80° C., one portion of initiator was added, and the feeds of lauryl methacrylate and Vinyl pyrrolidone were commenced. Lauryl methacrylate (108 g) was fed over an hour, while Vinyl pyrrolidone (27 g) combined with (50 g) t-butanol was fed over 2 hours. Every hour a fresh portion of initiator was added. Upon completion of the feeds, the reaction was held for four more hours with additional shots of initiator added hourly. The reaction was then heated to 90° C. and held for 12 hours. The resultant polymer solution was cooled to 35° C. and discharged from the reactor. A viscous water-white solution of polymer is obtained. Polymer solution is solvent exchanged by stripping t-butanol reaction solvent and replacing with applications friendly delivery solvent, most preferably isohexadecane.

Oil Soluble Compositions

TABLE 1
Physical Characteristics of Polymer Compositions

| | Composition(s) | | | | | |
|---|---|---|---|---|---|---|
| | 1 | | 2 | | 3 | |
| Solvent Used | t-Butanol | | t-Butanol | | t-Butanol | |
| LMA/Hydroxyethyl | 80/20 | | 60/40 | | 40/60 | |
| pyrrolidone methacrylate | | | | | | |
| Hydroxyethyl pyrrolidone | ND | [LC] | <100 | ppm | <100 | ppm |
| methacrylate in ppm | | | | | | |
| LMA in % | 0.27 | [LC] | <100 | ppm | <100 | ppm |
| % Solids | 52.4% | | 50% | | 50% | |
| Soluble in Mineral Oil | Yes | | Yes | | Yes | |
| Molecular Weight | 154K | [4.6] | 180K | [3.4] | 200K | [3.0] |
| Monomer Ratio | 74/26 | | 50/50 | | 75/25 | |
| Tg | >20° | C. | >20° | C. | >20° | C. |

Polymers Incorporated into Shampoo System

Odor intensity was judged by the scoring system listed in the below Table 2:

TABLE 2
Scoring System

| Score | Intensity |
|---|---|
| 1.0 | No Smell |
| 2.0 | Weak |
| 3-3.5 | Medium |
| 4.0 | Moderately Strong |
| 5.0 | Strong |

The polymers prepared according to process described in examples 2-11 were incorporated into the shampoo systems to test their effect on the physical properties of shampoo system and for fragrance deposition efficacy on human hair.

TABLE 3
Efficacy of polymers to deposit long lasting fragrance effect

| | | | | Fragrance |
|---|---|---|---|---|
| | | Monomer | Molecular | Delivery |
| Example | Copolymer | ratio | weight/PDI | Effect |
| 1 | LMA/HEPMA | 80/20 | 620,000/6.5 | 5 |
| 2 | LMA/HEPMA | 80/20 | 721,000/15.5 | 5 |
| 3 | LMA/HEPMA | 80/20 | 360,000/5.4 | 3 |
| 4 | LMA/HEPMA | 80/20 | 538,000/7.5 | 3 |
| 5 | LMA/HEPMA | 80/20 | 212,000/3.6 | 3 |
| 6 | LMA/HEPMA | 80/20 | 152,000/4.4 | 3 |
| 7 | LMA/HEPMA | 80/20 | 77,500/3.8 | 3 |
| 8 | LMA/HEPMA | 60/40 | 180,000/3.4 | 2 |
| 9 | LMA/HEPMA | 40/60 | 200,000/3.0 | 2 |
| 10 | LMA/HEPMA | 90/10 | 315,000/8.1 | 1 |
| 11 | LMA/HEPMA | 10/90 | 19,800/14 | 1 |
| A | Lauryl | 100 | 542,000/6.2 | 2 |
| | methacrylate | | | |
| 13 | Stearyl | 80/20 | 289,000/7.7 | 1 |
| | methacrylate/ | | | |
| | HEPMA | | | |
| 14 | Ethylhexyl | 80/20 | 256,000/4.9 | 1 |
| | methacrylate | | | |
| | (C8 branched)/ | | | |
| | HEPMA | | | |
| 15 | Ethylhexyl | 80/20 | 101,000/4.2 | 1 |
| | methacrylate | | | |
| | (C8 branched)/ | | | |
| | HEPMA | | | |
| 16 | Ethylhexyl | 80/20 | 148,000/4.4 | 1 |
| | methacrylate | | | |
| | (C8 branched)/ | | | |
| | HEPMA | | | |
| 17 | Ethylhexyl | 80/20 | 142,000/4.3 | 1 |
| | methacrylate | | | |
| | (C8 branched)/ | | | |
| | HEPMA | | | |
| B | LMA/VP | 80/20 | 626.000/6.7 | 2 |

LMA—Lauryl Methacrylate;
HEPMA—Hydroxyethyl pyrrolidone methacrylate

Based on the experiments and results as shown in Table 3, the the preferred compositions with the highest fragrance intensity and longevity have a molecular weight in the order of over 600,000, and are composed of an 80/20 ratio of LMA/Hydroxyethyl Pyrrolidone methacrylate.

Shampoo Formulations with and without LMA/HEPMA Polymer

Shampoo formulations were prepared, a shampoo with a copolymer consisting of 80/20 ratio of Lauryl methacrylate/hydroxyethyl pyrrolidone methacrylate copolymer at a level of 0.5% active, and the other one is a control shampoo without polymer. Formula or compositions are provided in the below Table 4.

TABLE 4
Shampoo Formulations for Panel Study

| | | Without | With |
|---|---|---|---|
| | Component | Polymer | Polymer |
| Phase A | Water | q.s. | q.s. |
| | Sodium laureth | 48.00 | 48.00 |
| | sulfate | | |
| | Cocamidopropyl | 10.34 | 10.34 |
| | Betaine | | |
| Phase B | Conditioning | 0.00 | 1.50 |
| | Polymer | | |
| Phase C | LMA/HEPMA 80/20 | 0.00 | 0.50 |
| Phase D | Orchid (Fragrance) | 0.50 | 0.50 |
| | Sodium Chloride | 0.50 | 3.50 |
| | | 100.00 | 100.00 |
| | pH | 6.48 | 6.81 |
| | Viscosity | 7,792 | 1,896 |
| | Appearance | Clear | Hazy, yellow |

Phase A was added into main container. Contents were mixed with propeller blade and heated to 45° C. Conditioning polymer of Phase B was added and mixed until solution becomes uniform. Mix until uniform. Temperature was maintained at 45° C. Observations were made during incorporation as well as when mixed. Later experimental hydrophobic polymer of Phase C was added and mixed until uniform. Temperature was maintained at 45° C. and then cooled to 25-30° C. Fragrance and other auxiliary ingredients such as sodium chloride of Phase D were added at 25-30° C. and mixed until uniform. Initial pH, viscosity, and physical observations of batch were noted.

Mannequin Head Study to Show Fragrance Long-Lastingness

Shampoo (5 ml) prepared as per formulas mentioned in Table 3 were combined with water at 37° C. and applied to mannequin head hair for 1-2 minutes. Rinse until hair is clear of foam. Hair was left to air dry and a trained professional fragrance evaluator judged the odor intensity and odor character both initially after the hair was dry and after various periods of time. Odor character is judged by someone who can discern the subtle components of the fragrance blend such as someone who has training in the art of fragrance evaluation. Results are tabulated below:

TABLE 5
Odor Intensity/Character Test Results

| | | Intensity | Intensity | Character | Character |
|---|---|---|---|---|---|
| | | Without | With | Without | With |
| Hair Status | Time | Polymer | Polymer | Polymer | Polymer |
| Wet | 10:30 | 2 | 2 | soft floral | soft floral |
| Semi Dry | 11:00 | 2 | 2 | soft floral | soft floral |
| Initial Dry | 12:00 | 2 | 4 | soft floral | sharp, clean |
| | | | | | floral |
| 1 Hour | 1:00 | 2 | 4 | soft well- | sharp, clean |
| | | | | rounded | floral |
| | | | | floral | |
| 2 Hour | 2:00 | 2.5 | 4 | soft well- | sharp, clean |
| | | | | rounded | floral |
| | | | | floral | |
| 4 Hour | 4:00 | 2.5 | 4 | soft well- | sharp, clean |
| | | | | rounded | floral |
| | | | | floral | |
| 24 Hour | 9:00 | 2 | 3 | soft well- | sharp, clean |
| | | | | rounded | floral |
| | | | | floral | |

Odor intensity and more importantly, odor character was retained on the mannequin head shampooed with the formula having the copolymer of this application more effectively than without the copolymer showing the efficacy of the polymer to not only deliver fragrance to the hair but to improve its long-lasting intensity and character.

Fragrance Delivery Evaluation on Natural Undamaged Hair

Shampoo formulations were prepared comprising polymer LMA/HEPMA and conditioning polymer in different concentrations illustrated below in Table 6.

TABLE 6
Shampoo Formulations for Panel Study

| | Component | 1 | |
|---|---|---|---|
| Phase A | Water | 21.66 | 43.32 |
| | Sodium Laureth Sulfate - 2 | 48.00 | 96.00 |
| | Cocamidopropyl Betaine | 10.34 | 20.68 |
| Phase B | Conditioning Polymer | 5.00 | 10.00 |
| | (30%) | | |
| Phase C | LMA/HEPMA polymer | 1.67 | 3.34 |
| | (30%) 80/20 | | |
| Phase D | Water | 9.13 | 18.26 |
| | Orchid (Fragrance) | 0.50 | 1.00 |
| | | 3.50 | 7.00 |
| | | 0.20 | 0.40 |
| | | 100.00 | 200.00 |
| | pH | | 6.69 |
| | Viscosity (Sp#5, | | 2504 |
| | 50 rpm, RT) | | |
| | Appearance | | Hazy, |
| | | | white |

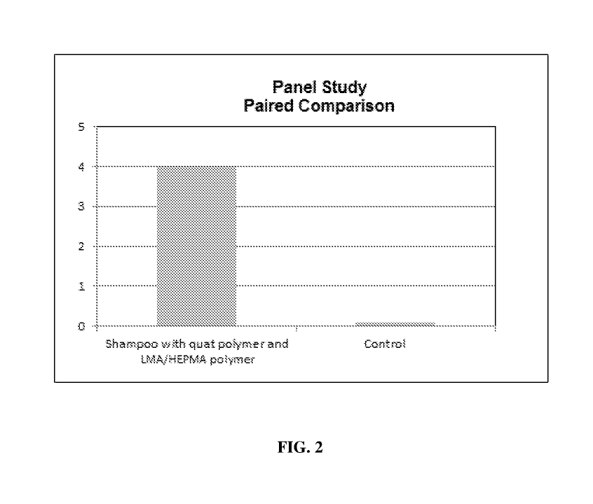

Formulations prepared as per Table 6 were subjected to humar hair application. Hair Swatches are prepared by applying the shampoo (1 cc) over 6.5 inch loose undamaged natural human hair tresses. Shampoo was washed for 1 minute under 37° C. and rinsed for 30 seconds. Hair tresses composed of natural undamaged hair were tested by a group of trained fragrance evaluators. The tresses are then air dried and fragrance intensity is evaluated over the course of one day or 8 hours. The tresses are then air dried and fragrance intensity is evaluated over the course of one day or 8 hours. Four out of four expert panelists were able to discern an intensity difference both initially as well as over the eight-hour time span showing the efficacy of the hydrophobic polymer on fragrance long-lastingness. Results are depicted graphically in FIG. 2.

Formulations with Alternate Fragrances

To illustrate the compatibility, range of the hydrophobic polymer with other fragrances, Orchid fragrance was substituted with other fragrances. These are illustrated in the below Table 7.

TABLE 7
Shampoo Formulations with Alternate Fragrances

| | Component | 1 | | 2 | | 3 | |
|---|---|---|---|---|---|---|---|
| Phase A | Water | 21.66 | 86.64 | 21.66 | 86.64 | 21.66 | 86.64 |
| | Sodium Laureth Sulfate | 48.00 | 192.00 | 48.00 | 192.00 | 48.00 | 192.00 |
| | (25%) | | | | | | |
| | Cocamidopropyl Betaine | 10.34 | 41.36 | 10.34 | 41.36 | 10.34 | 41.36 |
| | (29%) | | | | | | |
| | | | 0.00 | | 0.00 | | 0.00 |
| Phase B | Conditioning Polymer | 5.00 | 20.00 | 5.00 | 20.00 | 1.67 | 6.68 |
| | (30%) | | | | | | |
| | | | 0.00 | | 0.00 | | 0.00 |
| Phase C | LMA/HEPMA (30%) 80/20 | 1.67 | 6.68 | 1.67 | 6.68 | 1.67 | 6.68 |
| | | | 0.00 | | 0.00 | | 0.00 |
| Phase D | Water | 9.13 | 36.52 | 9.13 | 36.52 | 12.46 | 49.84 |
| | Fragrance Robertet R16- | 0.50 | 2.00 | 0.00 | 0.00 | 0.50 | 2.00 |
| | 2476 | | | | | | |
| | Fragrance Robertet R16- | 0.00 | 0.00 | 0.50 | 2.00 | 0.00 | 0.00 |
| | 2551 | | | | | | |
| | NaCl | 3.50 | 14.00 | 3.50 | 14.00 | 3.50 | 14.00 |
| | Propylene Glycol (and) | 0.20 | 0.80 | 0.20 | 0.80 | 0.20 | 0.80 |
| | Diazolidinyl Urea (and) | | | | | | |
| | Iodopropynyl | | | | | | |
| | Butylcarbamate | | | | | | |
| | (Preservative) | | | | | | |
| | | 100.00 | 400.00 | 100.00 | 400.00 | 100.00 | 400.00 |
| | pH | | 6.57 | | 6.61 | | 6.26 |
| | Viscosity (Sp#5, 50 rpm, | | 5,432 | | 2,600 | | 2,024 |
| | RT) | | | | | | |

| | | | |
|---|---|---|---|
| Appearance | hazy, yellow | hazy, yellow | hazy, yellow |

The above formulas showed good integrity from a physical standpoint as well as efficacy during on hair fragrance long-lastingness tests on hair swatches.

Shampoo Formulations using LMA/HEPMA 80/20 with Alternate molecular weights:

LMA/HEPMA copolymer having different molecular weights were prepared and analyzed for fragrance deposition.

TABLE 8
Shampoo Formulations with LAM/HEPMA
80/20 (Alternate Molecuar Weights)

| | Component | 1 | | 2 | |
|---|---|---|---|---|---|
| Phase A | Water | 21.66 | 43.32 | 21.66 | 43.32 |
| | Sodium Laureth | 48.00 | 96.00 | 48.00 | 96.00 |
| | Sulfate (25%) | | | | |
| | Cocamidopropyl | 10.34 | 20.68 | 10.34 | 20.68 |
| | Betaine (29%) | | | | |
| Phase B | Conditioning | 5.00 | 10.00 | 5.00 | 10.00 |
| | Polymer (30%) | | | | |
| Phase C | LMA/HEPMA | 1.11 | 2.22 | 0.00 | 0.00 |
| | 80/20 (45%) | | | | |
| | LMA/HEPMA | 0.00 | 0.00 | 1.11 | 2.22 |
| | 80/20 (45%) | | | | |
| Phase D | Water | 9.69 | 19.38 | 9.69 | 19.38 |
| | Orchid | 0.50 | 1.00 | 0.50 | 1.00 |
| | (Fragrance) | | | | |
| | NaCl | 3.50 | 7.00 | 3.50 | 7.00 |
| | Propylene | 0.20 | 0.40 | 0.20 | 0.40 |
| | Glycol (and) | | | | |
| | Diazolidinyl | | | | |
| | Urea (and) | | | | |
| | Iodopropynyl | | | | |
| | Butylcarbamate | | | | |
| | (Preservative) | | | | |
| | | 100.00 | 200.00 | 100.00 | 200.00 |
| | pH | | 6.22 | | 6.56 |
| | Viscosity (Sp#5, | | 54,480 | | 55,280 |
| | 50 rpm, RT) | | | | |

| | | |
|---|---|---|
| Appearance | translucent, | translucent, |
| | yellow | yellow |

Shampoo Formulations Using LMA/HEPMA 80/20 with Alternate Concentrations:

TABLE 9
Formulations with LMA/HEPMA 80/20 ratio
having alternate concentrations

| | Component | 1 | | 2 | |
|---|---|---|---|---|---|
| Phase A | Water | 21.66 | 43.32 | 21.66 | 43.32 |
| | Sodium Laureth | 48.00 | 96.00 | 48.00 | 96.00 |
| | Sulfate (25%) | | | | |
| | Cocamidopropyl | 10.34 | 20.68 | 10.34 | 20.68 |
| | Betaine (29%) | | | | |
| Phase B | Conditioning | 5.00 | 10.00 | 5.00 | 10.00 |
| | Polymer (30%) | | | | |
| Phase C | LMA/HEPMA | 1.25 | 2.50 | 0.00 | 0.00 |
| | (80/20) (40%) | | | | |
| | LMA/HEPMA | 0.00 | 0.00 | 1.43 | 2.86 |
| | (80/20) (35%) | | | | |
| Phase D | Water | 9.55 | 19.10 | 9.37 | 18.74 |
| | Orchid | 0.50 | 1.00 | 0.50 | 1.00 |
| | (Fragrance) | | | | |
| | NaCl | 3.50 | 7.00 | 3.50 | 7.00 |
| | Propylene | 0.20 | 0.40 | 0.20 | 0.40 |
| | Glycol (and) | | | | |
| | Diazolidinyl | | | | |
| | Urea (and) | | | | |
| | Iodopropynyl | | | | |
| | Butylcarbamate | | | | |
| | (Preservative) | | | | |
| | | 100.00 | 200.00 | 100.00 | 200.00 |
| | pH | | 6.60 | | 6.60 |
| | Viscosity (Sp#5, | | 6600 | (5@10 rpm) | 35560 |
| | 50 rpm, RT) | | | | |
| | Appearance | | Hazy | Hazy | |

Shampoo Formulations Using LMA/HEPMA 80/20 with Alternate Fragrances:

TABLE 10
Formulations having Polymers with Other Fragrances

| | Component | 1 | | 2 | |
|---|---|---|---|---|---|
| Phase A | Water | 21.66 | 43.32 | 21.66 | 43.32 |
| | Sodium Laureth | 48.00 | 96.00 | 48.00 | 96.00 |
| | Sulfate (25%) | | | | |
| | Cocamidopropyl | 10.34 | 20.68 | 10.34 | 20.68 |
| | Betaine (29%) | | | | |
| Phase B | Conditioning | 5.00 | 10.00 | 5.00 | 10.00 |
| | Polymer (30%) | | | | |
| Phase C | LMA/HEPMA | 1.11 | 2.22 | — | — |
| | 80/20 (45%) | | | | |
| | LMA/HEPMA | — | — | 1.25 | 2.50 |
| | 80/20 (40%) | | | | |
| Phase D | Water | 9.69 | 19.38 | 9.55 | 19.10 |
| | Fragrance | 0.50 | 1.00 | 0.50 | 1.00 |
| | Robertet R16-2476 | | | | |
| | NaCl | 3.50 | 7.00 | 3.50 | 7.00 |
| | Propylene Glycol | 0.20 | 0.40 | 0.20 | 0.40 |
| | (and) Diazolidinyl | | | | |
| | Urea (and) | | | | |
| | Iodopropynyl | | | | |
| | Butylcarbamate | | | | |
| | (Preservative) | | | | |
| | | 100.00 | 200.00 | 100.00 | 200.00 |
| | pH | | 6.62 | | 6.75 |
| | Viscosity (Sp#5, 10 rpm, RT) | | 27,120 | | 36,000 |
| | Appearance | Clear | | Hazy | |

Shampoo Formulations Using LMA/HEPMA in Alternate Ratios

TABLE 11
Formulations with Polymers having different LMA and HEPMA Ratios

| | Component | 1 | 2 | 3 | 4 | 5 |
|---|---|---|---|---|---|---|
| Phase A | Water | 21.66 | 21.66 | 21.66 | 21.66 | 21.66 |
| | Sodium Laureth Sulfate | 48.00 | 48.00 | 48.00 | 48.00 | 48.00 |
| | (25%) | | | | | |
| | Cocamidopropyl Betaine | 10.34 | 10.34 | 10.34 | 10.34 | 10.34 |
| | (29%) | | | | | |
| Phase B | Conditioning Polymer | 5.00 | 5.00 | 5.00 | 5.00 | 5.00 |
| | (30%) | | | | | |
| Phase C | LMA/HEPMA 80/20 | 1.25 | — | — | — | — |
| | (40%) | | | | | |
| | LMA/HEPMA 75/25 | — | 1.25 | — | — | — |
| | (40%) | | | | | |
| | LMA/HEPMA 80/20 | — | — | 1.67 | — | — |
| | (30%) | | | | | |
| | LMA/HEPMA 80/20 | — | — | — | 2.50 | — |
| | (20%) | | | | | |
| | LMA/HEPMA 80/20 | — | — | — | — | 2.50 |
| | (20%) | | | | | |
| | Water | 9.55 | 9.55 | 9.13 | 8.30 | 8.30 |
| Phase D | Fragrance | 0.50 | 0.50 | 0.50 | 0.50 | 0.50 |
| | Robertet R16-2476 | | | | | |
| | NaCl | 3.50 | 3.50 | 3.50 | 3.50 | 3.50 |
| | Propylene Glycol (and) | 0.20 | 0.20 | 0.20 | 0.20 | 0.20 |
| | Diazolidinyl Urea (and) | | | | | |
| | Iodopropynyl | | | | | |
| | Butylcarbamate | | | | | |
| | (Preservative) | | | | | |
| | | 100.00 | 100.00 | 100.00 | 100.00 | 100.00 |
| | pH | 6.63 | 6.63 | 6.79 | 6.66 | 6.63 |
| | Viscosity (Sp#5, 50 rpm, | (@ 10 rpm) | (@ 5 rpm) | 5648 | 344 | 3544 |
| | RT) | 34360 | 59000 | | | |
| | Appearance | Very | Clear | Clear | Clear | Clear |
| | | Hazy | | | | |

Shampoo Formulations Using LMA/Vinyl Pyrrolidone 80/20

Copolymer replacing HEPMA with vinyl pyrrolidone was tested for fragrance delivery.

TABLE 12
Formulation having LMA/VP copolymer

| | Component | 1 | |
|---|---|---|---|
| Phase A | Water | 21.66 | 43.32 |
| | Sodium Laureth | 48.00 | 96.00 |
| | Sulfate (25%) | | |
| | Cocamidopropyl | 10.34 | 20.68 |
| | Betaine (29%) | | |
| Phase B | Conditioning | 5.00 | 10.00 |
| | Polymer (30%) | | |
| Phase C | LMA/VP | 1.25 | 2.50 |
| | 80/20 (40%) | | |
| Phase D | Water | 9.55 | 19.10 |
| | Orchid (Fragrance) | 0.50 | 1.00 |
| | NaCl | 3.50 | 7.00 |
| | Propylene | 0.20 | 0.40 |
| | Glycol (and) | | |
| | Diazolidinyl | | |
| | Urea (and) | | |
| | Iodopropynyl | | |
| | Butylcarbamate | | |
| | (Preservative) | | |
| | | 100.00 | 200.00 |
| | pH | | 6.69 |
| | Viscosity | | 39,080 |
| | Appearance | opaque, | |
| | | white | |

The shampoo comprising LMA/VP copolymer resulted in an opaque product and showed reduced efficacy in fragrance evaluation tests.

Shampoos with Commercial Conditioning Polymers

Most shampoo systems on the market have one or more types of conditioning polymers in order to aid in wet and dry combing and feel attributes after washing. The series of formulas made as illustrated in Tables 13 through 16 are made to determine the compatibility of a preferred hydrophobic polymer, namely LMA/HEPMA 80/20 of high molecular weight with a host of common commercially available cationic and amphoteric type polymeric conditioning polymers. These polymers represent a full spectrum of molecular weights, charge densities, and polymer backbone chemistries. The physical properties of the shampoos indicate that the preferred hydrophobic polymer is compatible with these representative conditioning polymers.

TABLE 13
Formulations with Commercial Conditioning Polymers

| | Component | 1 | | 2 | |
|---|---|---|---|---|---|
| Phase A | Water | 21.66 | 43.32 | 21.66 | 43.32 |
| | Sodium Laureth | 48.00 | 96.00 | 48.00 | 96.00 |
| | Sulfate (25%) | | | | |
| | Cocamidopropyl | 10.34 | 20.68 | 10.34 | 20.68 |
| | Betaine | | | | |
| Phase B | Polyquaternium- | 3.75 | 7.50 | 0.00 | 0.00 |
| | 22 (40%) | | | | |
| | Polyacryl- | 0.00 | 0.00 | 7.50 | 15.00 |
| | amidopropyl | | | | |
| | trimonium | | | | |
| | Chloride (20%) | | | | |
| Phase C | LMA/HEPMA | 1.67 | 3.34 | 1.67 | 3.34 |
| | 80/20 (30%) | | | | |
| Phase D | Water | 10.38 | 20.76 | 6.63 | 13.26 |
| | Orchid (Fragrance) | 0.50 | 1.00 | 0.50 | 1.00 |
| | NaCl | 3.50 | 7.00 | 3.50 | 7.00 |
| | Propylene | 0.20 | 0.40 | 0.20 | 0.40 |
| | Glycol (and) | | | | |
| | Diazolidinyl | | | | |
| | Urea (and) | | | | |
| | Iodopropynyl | | | | |
| | Butylcarbamate | | | | |
| | (Preservative) | | | | |
| | | 100.00 | 200.00 | 100.00 | 200.00 |
| | pH | | | | 5.50 |
| | Viscosity | | | | 13250 |

| | |
|---|---|
| Appearance | Opaque, white |

TABLE 14
Formulations with Commercial Conditioning
Polymers with Alternate Fragrances

| | Component | 1 | |
|---|---|---|---|
| Phase A | Water | 21.66 | 43.32 |
| | Sodium Laureth Sulfate (25%) | 48.00 | 96.00 |
| | Cocamidopropyl Betaine (29%) | 10.34 | 20.68 |
| Phase B | Polyacrylamidopropyl | 2.50 | 5.00 |
| | trimonium Chloride (20%) | | |
| Phase C | LMA/HEPMA 80/20 (30%) | 1.67 | 3.34 |
| Phase D | Water | 11.63 | 23.26 |
| | Fragrance (R16-2476) | 0.50 | 1.00 |
| | NaCl | 3.50 | 7.00 |
| | Propylene Glycol (and) | 0.20 | 0.40 |
| | Diazolidinyl Urea (and) | | |
| | Iodopropynyl Butylcarbamate | | |
| | (Preservative) | | |
| | | 100.00 | 200.00 |
| | pH | | 5.70 |
| | Viscosity | | 6,208 |
| | Appearance | Very | |
| | | Hazy | |

TABLE 15
Formulations with Commercial Conditioning Polymers

| | Component | 1 | 2 | 3 | 4 | 5 | 6 | 7 |
|---|---|---|---|---|---|---|---|---|
| Phase A | Water | 35.59 | 35.59 | 35.59 | 35.59 | 35.59 | 35.59 | 35.59 |
| | Guar HPTC | 0.20 | — | — | — | — | — | — |
| | Guar HPTC | — | 0.20 | — | — | — | — | — |
| | Guar HPTC | — | — | 0.20 | — | — | — | — |
| | Guar HPTC | — | — | — | 0.20 | — | — | — |
| | Guar HPTC | — | — | — | — | 0.20 | — | — |
| | Guar HPTC | — | — | — | — | — | 0.20 | — |
| | APTAC/Acrylamide | — | — | — | — | — | — | 0.20 |
| | 10% NaOH | qs | qs | qs | qs | qs | qs | qs |
| Phase B | Cocamidopropyl | 10.34 | 10.34 | 10.34 | 10.34 | 10.34 | 10.34 | 10.34 |
| | Betaine (29%) | | | | | | | |
| | Sodium laureth | 48.00 | 48.00 | 48.00 | 48.00 | 48.00 | 48.00 | 48.00 |
| | Sulfate (25%) | | | | | | | |
| Phase C | LMA/HEPMA | 1.67 | 1.67 | 1.67 | 1.67 | 1.67 | 1.67 | 1.67 |
| | 80/20 (30%) | | | | | | | |
| Phase D | Orchid (Fragrance) | 0.50 | 0.50 | 0.50 | 0.50 | 0.50 | 0.50 | 0.50 |
| | NaCl | 3.50 | 3.50 | 3.50 | 3.50 | 3.50 | 3.50 | 3.50 |
| | Propylene Glycol | 0.20 | 0.20 | 0.20 | 0.20 | 0.20 | 0.20 | 0.20 |
| | (and) Diazolidinyl | | | | | | | |
| | Urea (and) | | | | | | | |
| | Iodopropynyl | | | | | | | |
| | Butylcarbamate | | | | | | | |
| | (Preservative) | | | | | | | |
| | Citrc Acid | | | | | | | |
| | | 100.00 | 100.00 | 100.00 | 100.00 | 100.00 | 100.00 | 100.00 |
| | pH | 6.12 | 5.95 | 6.07 | 6.08 | 6.08 | 6.11 | 6.05 |
| | Viscosity | 5,464 | 4,744 | 1,904 | 3,264 | 184.0 | 7,920 | 4,736 |
| | Appearance | hazy | hazy | hazy | hazy | hazy, | hazy | hazy, |
| | | | | | | particulate | | particulate |

HPTC—Hydroxypropyltrimonium chloride;
APTAC—Acrylamidopropyl trimonium chloride

Formulation Variants

A preferred composition, namely the LMA/HEPMA, was added to shampoos with reduced washing actives to explore the versatility of this compound to be compatible in a more cost effective retail type of shampoo. These are illustrated in Tables 16. As expected, viscosities were lower, but someone acquainted with shampoo formulations can add auxiliary ingredients such as thickening polymers to compensate. The results demonstrate a compatibility in a system with reduced washing actives.

TABLE 16
Shampoos with Reduced Washing Actives with Alternate Fragrance

| | Component | 1 | | 2 | | 3 | | 4 | |
|---|---|---|---|---|---|---|---|---|---|
| Phase A | Water | 40.86 | 122.58 | 41.42 | 124.26 | 41.28 | 123.84 | 42.53 | 127.59 |
| | Sodium Laureth | 31.37 | 94.11 | 31.37 | 94.11 | 31.37 | 94.11 | 31.37 | 94.11 |
| | Sulfate (25%) | | | | | | | | |
| | Cocamidopropyl | 6.90 | 20.70 | 6.90 | 20.70 | 6.90 | 20.70 | 6.90 | 20.70 |
| | Betaine (29%) | | | | | | | | |
| Phase B | Conditioning | 5.00 | 15.00 | 5.00 | 15.00 | 5.00 | 15.00 | 5.00 | 15.00 |
| | Polymer (30%) | | | | | | | | |
| Phase C | LMA/HEPMA | 1.67 | 5.01 | 0.00 | 0.00 | — | 0.00 | 0.00 | 0.00 |
| | 80/20 (30%) | | | | | | | | |
| | LMA/HEPMA | 0.00 | 0.00 | 1.11 | 3.33 | — | 0.00 | 0.00 | 0.00 |
| | 80/20 (45%) | | | | | | | | |
| | LMA/HEPMA | 0.00 | 0.00 | 0.00 | 0.00 | 1.25 | 3.75 | 0.00 | 0.00 |
| | 80/20 (40%) | | | | | | | | |
| Phase D | Water | 10.00 | 30.00 | 10.00 | 30.00 | 10.00 | 30.00 | 10.00 | 30.00 |
| | Fragrance | 0.50 | 1.50 | 0.50 | 1.50 | 0.50 | 1.50 | 0.50 | 1.50 |
| | (Robertet | | | | | | | | |
| | R16-2476) | | | | | | | | |
| | NaCl | 3.50 | 10.50 | 3.50 | 10.50 | 3.50 | 10.50 | 3.50 | 10.50 |
| | Propylene | 0.20 | 0.60 | 0.20 | 0.60 | 0.20 | 0.60 | 0.20 | 0.60 |
| | Glycol (and) | | | | | | | | |
| | Diazolidinyl | | | | | | | | |
| | Urea (and) | | | | | | | | |
| | Iodopropynyl | | | | | | | | |
| | Butylcarbamate | | | | | | | | |
| | (Preservative) | | | | | | | | |
| | | 100.00 | 300.00 | 100.00 | 300.00 | 100.00 | 300.00 | 100.00 | 300.00 |
| | pH | 8.51 | | 8.59 | | 8.60 | | 8.64 | |
| | pH adjusted | 6.64 | | 6.87 | | 6.79 | | 6.76 | |
| | with 10% citirc | | | | | | | | |
| | acid | | | | | | | | |
| | Viscosity | 32.0 | | 360.0 | | 1,200 | | 7,992 | |
| | (Sp#5, 50 rpm, | | | | | | | | |
| | RT) | | | | | | | | |
| | Appearance | Hazy | | Clear | | Very | | Clear | |
| | | | | | | hazy | | | |

Compatibility Studies of Hydrophobic Polymer with Other Fragrances

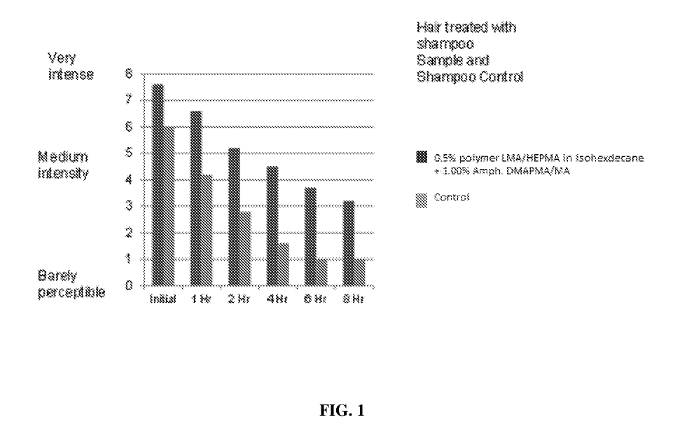

Experiment was performed to demonstrate effective delivery of fragrance compounds with and without copolymer of the present application. Particularly, the fragrance delivery experiments were performed for 0.5% of LMA/Hydroxyethyl pyrrolidone methacrylate in isohexadecane having 1.0% of DMAPMA/MA polymer, and it was observed that the LMA/Hydroxyethyl pyrrolidone methacrylate copolymer is capable of delivering the fragrance that lasts for 8 hours of duration with medium intensity. The delivery of fragrance for the copolymer was at its peak or intense levels during initial stage of shampooing and maintained to have medium level of fragrance delivery even at 8 hours completion, and whereas, the control sample without the copolymer demonstrated significantly less fragrance delivery as compared to copolymer samples right from initial level to 8 hrs completion stage. Further, control samples demonstrated barely perceptible level of fragrance delivery during 4-8 hrs stage while copolymer sample of this application is capable of delivering medium level fragrance delivery during 4-8 hrs stage (FIG. 1).

While this invention has been described in detail with reference to certain preferred embodiments, it should be appreciated that the present invention is not limited to those precise embodiments. Rather, in view of the present disclosure, many modifications and variations would present themselves to those skilled in the art without departing from the scope and spirit of this invention.

What is claimed is:

1. An oil soluble composition comprising a copolymer having repeating units of:
i. about 0.1-99.9 wt. % of at least one monomer selected from at least one hydrophobically modified (alk) acrylate moiety; and
ii. about 0.1-99.9 wt. % of at least one monomer derived from at least one functionalized or unfunctionalized acryloyl moiety and at least one lactam moiety.

2. The oil soluble composition according to claim 1, wherein said hydrophobically modified (alk) acrylate moiety is an (alk) acrylate comprising a straight or branched C₈-C₃₀ functionalized or unfunctionalized alkyl moiety.

3. The oil soluble composition according to claim 2, wherein said straight or branched C₈-C₃₀ alkyl moiety is selected from the group consisting of lauryl methacrylate (LMA), 2-ethylhexyl acrylate, 1,1,3,3-tetramethylbutyl acrylate, 1,1-dimethylhexyl acrylate, 6-methylheptyl acrylate, 7-methyloctyl acrylate, 2-propylheptyl acrylate, 8-methylnonyl acrylate, 9-methyldecyl acrylate, 10-methylundecyl acrylate, 11-methyldodecyl acrylate, 12-methyltridecyl acrylate, 13-methyltetradecyl acrylate, 14-methylpentadecyl acrylate, 15-methylhexadecyl acrylate, stearyl methacrylate, 16-methylheptadecyl acrylate, 17-methyloctadecyl acrylate, 2-ethylhexyl methacrylate, 1,1,3,3-tetramethylbutyl methacrylate, 1,1-dimethylhexyl methacrylate, 6-methylheptyl methacrylate, 7-methyloctyl methacrylate, 2-propylheptyl methacrylate, 8-methylnonyl methacrylate, 9-methyldecyl methacrylate, 10-methylundecyl methacrylate, 11-methyldodecyl methacrylate, 12-methyltridecyl methacrylate, 13-methyltetradecyl methacrylate, 14-methylpentadecyl methacrylate, 15-methylhexadecyl methacrylate, 16-methylheptadecyl methacrylate, 17-methyloctadecyl methacrylate, and combinations thereof.

4. The oil soluble composition according to claim 1, wherein said monomer derived from at least one functionalized or unfunctionalized acryloyl moiety and at least one lactam moiety is selected from compounds having the following structures of (a1)-(a8):

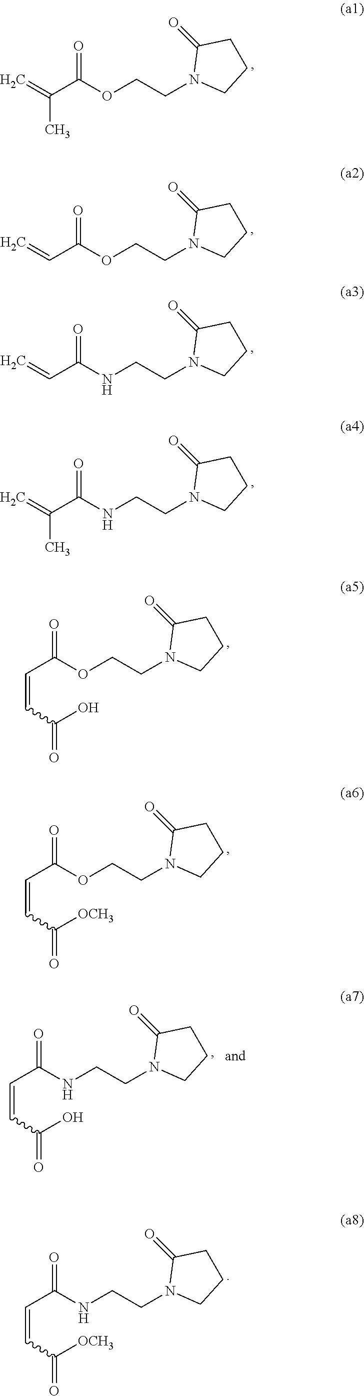

5. The oil soluble composition according to claim 1, wherein said copolymer is selected from compounds having the following structures: 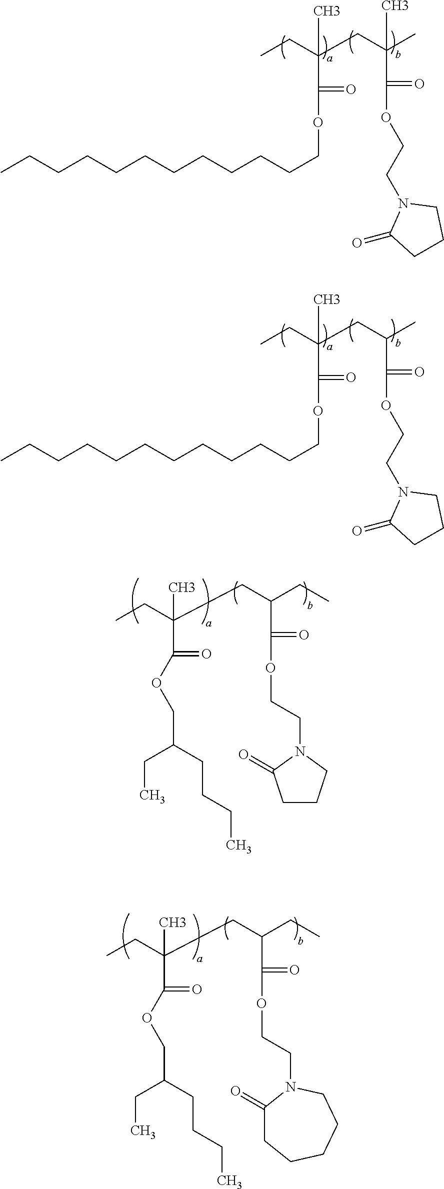 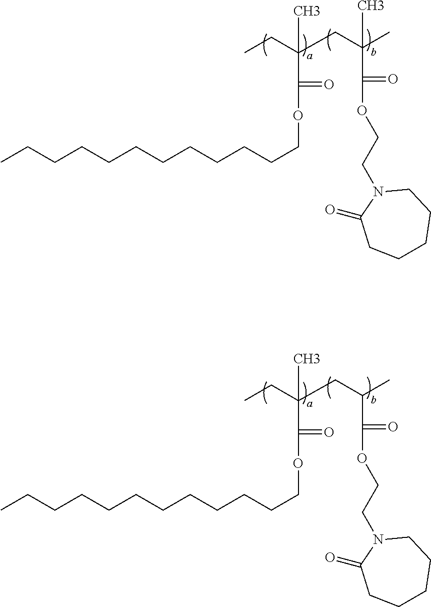
wherein each a, and b is an independently selected value ranging from about 0.1 to about 99.9 percent by weight of the polymer, with the provision that the sum of a and b for each polymer equals 100 weight percent.

6. The oil soluble composition according to claim 1, wherein said copolymer optionally comprises repeating units derived from at least one crosslinking agent.

7. The oil soluble composition according to claim 1, wherein said composition is a personal care composition, skin care composition, oral care composition, cementing fluid composition, oilfield composition, construction composition, servicing fluid composition, gravel packing mud composition, fracturing fluid composition, completion fluid composition, workover fluid composition, spacer fluid composition, drilling mud composition, coating composition, household composition, industrial and institutional composition, pharmaceutical composition, food composition, biocide composition, adhesive composition, ink composition, polish composition, membrane composition, metal working fluid composition, plastic composition, textile composition, printing composition, lubricant composition, preservative composition, agrochemical composition, fabric care composition, laundry composition, surface cleaning composition, dish washing composition or wood-care composition.

8. A mineral oil soluble personal care composition comprising:
(a) about 0.1 to about 99.9 wt. % of a copolymer having repeating units of (i) from about 0.1-99.9 wt. % of at least one monomer selected from at least one hydrophobically modified (alk)acrylate moiety, and (ii) about 0.1-99.9 wt. % of at least one monomer derived from at least one functionalized or unfunctionalized acryloyl moiety and at least one lactam moiety; and
(b) about 0.1 to about 99.9 wt. % of at least one cosmetically acceptable excipient.

9. The personal care composition according to claim 8, wherein said personal care composition is a sun care composition, a face care composition, a lip care composition, an eye care composition, a skin care composition, a sunscreen composition, a body care composition, a nail care composition, an anti-aging composition, an insect repellant composition; an oral care composition, a deodorant composition, a hair care composition, a conditioning composition, a color cosmetic composition, a color-protection composition, a self-tanning composition, a fragrance composition or a foot care composition.

10. The personal care composition according to claim 8, wherein said cosmetically acceptable excipient is selected from the group consisting of fatty substances, gelling agents, thickeners, surfactants, moisturizers, emollients, hydrophilic or lipophilic active agent, antioxidants, sequestering agents, preserving agents, acidifying or basifying agents, fragrances, fillers, dyestuffs, emulsifying agents, solvents, UV-A or UV-B blocker/filters, plant extracts, moisturizers, proteins, peptides, neutralizing agents, solvents, silicones and reducing agents.

11. A mineral oil soluble fragrance composition comprising:
(a) about 0.1 to about 99.9 wt. % of a copolymer having repeating units of (i) about 0.1-99.9 wt. % of at least one monomer selected from at least one hydrophobically modified (alk)acrylate moiety, and (ii) about 0.1-99.9 wt. % of at least one monomer derived from at least one functionalized or unfunctionalized acryloyl moiety and at least one lactam moiety; and
(b) about 0.1 to about 99.9 wt. % of at least one cosmetically acceptable excipient.

12. The mineral oil soluble fragrance composition according to claim 11, wherein said cosmetically acceptable excipient is selected from the group consisting of fatty substances, gelling agents, thickeners, surfactants, moisturizers, emollients, hydrophilic or lipophilic active agent, antioxidants, sequestering agents, preserving agents, acidifying or basifying agents, fragrances, fillers, dyestuffs, emulsifying agents, solvents, UV-A or UV-B blocker/filters, plant extracts, moisturizers, proteins, peptides, neutralizing agents, solvents, silicones and/or reducing agents.

13. A delivery system for oil based functional ingredients comprising:
a. about 0.1 to about 99.9 wt. % of a copolymer having repeating units of: (i) from about 0.1-99.9 wt. % of at least one monomer selected from at least one hydrophobically modified (alk)acrylate moiety; and (ii) from about 0.1-99.9 wt. % of at least one monomer derived from at least one functionalized or unfunctionalized acryloyl moiety and at least one lactam moiety; and
b. about 0.1 to about 99.9 wt. % of at least one oil based functional ingredient.

14. The delivery system according to claim 13, wherein said functional ingredient is fragrance.

15. A fragrance delivery system for keratin based substrate comprising:
a. about 1 to 25 wt. % of an active ingredient fragrance;
b. about 75 to 99 wt. % of an emulsion concentrate comprising a copolymer having repeating units of: (i) from about 0.1-99.9 wt. % of at least one monomer selected from at least one hydrophobically modified (alk)acrylate moiety; and (ii) from about 0.1-99.9 wt. % of at least one monomer derived from at least one functionalized or unfunctionalized acryloyl moiety and at least one lactam moiety; and
c. water.

16. The fragrance delivery system according to claim 15, wherein said emulsion is a nano emulsion, micro emulsion or mini emulsion.

17. The fragrance delivery system according to claim 15, wherein said system releases fragrance gradually that lasts for at least 8 hours.

18. A composition for delivering fragrance from a shampoo for hair care comprising:
(a) 1 to 25% of an active ingredient fragrance;
(b) 75 to 99% of an emulsion concentrate comprising a copolymer having repeating units derived from: (i) from about 0.1-99.9 wt. % of at least one monomer selected from at least one hydrophobically modified (alk)acrylate moiety; and (ii) from about 0.1-99.9 wt. % of at least one monomer derived from at least one functionalized or unfunctionalized acryloyl moiety and at least one lactam moiety, and
(c) water.

19. The method according to claim 18, wherein said shampoo is 2-in-1 shampoo or 3-in-1 shampoo.
